SEQUENCE LISTING (106 sequences)

SEQ ID NO: 1 - protein, 24 aa, Homo sapiens
AKWDTANNPLYKEATSTFTNITYR

SEQ ID NO: 2 - protein, 26 aa, Homo sapiens
AKWDTANNPLYKEATSTFTNITYRGT

SEQ ID NO: 3 - protein, 18 aa, Homo sapiens
AKWDTVRDGAGRFLKSLV

SEQ ID NO: 4 - protein, 18 aa, Homo sapiens
AKWDTHYAQSLRKWNQPV

SEQ ID NO: 5 - protein, 19 aa, Homo sapiens
  VARIANT (2)..(3): Corresponding synthetic peptide may have modified lysine residues bearing an isotopic tag or isobaric tag
LKKANLQIDQINTDLNLER

SEQ ID NO: 6 - protein, 9 aa, Homo sapiens
  VARIANT (2)..(2): Corresponding synthetic peptide may have a modified lysine residue bearing an isotopic tag or isobaric tag
EKQLAAENR

SEQ ID NO: 7 - protein, 22 aa, Homo sapiens
  VARIANT (13, 21): Corresponding synthetic peptide may have modified lysine residues bearing an isotopic tag or isobaric tag
DELADEIANSSGKGALALEEKR

SEQ ID NO: 8 - protein, 23 aa, Homo sapiens
  VARIANT (21)..(21): Corresponding synthetic peptide may have a modified lysine residue bearing an isotopic tag or isobaric tag
INFDVNGYIVGANIETYLLEKSR

SEQ ID NO: 9 - protein, 10 aa, Homo sapiens
ALEATTEHIR

SEQ ID NO: 10 - protein, 13 aa, Homo sapiens
DPPSWSVLAGHSR

SEQ ID NO: 11 - protein, 17 aa, Homo sapiens
  VARIANT (4)..(4): Corresponding synthetic peptide may have a modified lysine residue bearing an isotopic tag or isobaric tag
VSEKVSHVLAALQAGNR

SEQ ID NO: 12 - protein, 14 aa, Homo sapiens
  VARIANT (6)..(6): Corresponding synthetic peptide may have a modified lysine residue bearing an isotopic tag or isobaric tag
LAQVAKAVTQALNR

SEQ ID NO: 13 - protein, 12 aa, Homo sapiens
  VARIANT (1)..(1): Corresponding synthetic peptide may have a modified lysine residue bearing an isotopic tag or isobaric tag
KFFYSDQNVDSR

SEQ ID NO: 14 - protein, 10 aa, Homo sapiens
  VARIANT (8)..(8): Corresponding synthetic peptide may have a modified lysine residue bearing an isotopic tag or isobaric tag
EAEAASIKIR

SEQ ID NO: 15 - protein, 19 aa, Homo sapiens
GILSGTSDLLLTFDEAEVR

SEQ ID NO: 16 - protein, 16 aa, Homo sapiens
  VARIANT (11)..(11): Corresponding synthetic peptide may have a modified lysine residue bearing an isotopic tag or isobaric tag
SLGEISALTSKLADLR

SEQ ID NO: 17 - protein, 15 aa, Homo sapiens
DPSASPGDAGEQAIR

SEQ ID NO: 18 - protein, 7 aa, Homo sapiens
  VARIANT (1)..(1): Corresponding synthetic peptide may have a modified lysine residue bearing an isotopic tag or isobaric tag
KLTSNLR

SEQ ID NO: 19 - protein, 18 aa, Homo sapiens
  VARIANT (10)..(10): Corresponding synthetic peptide may have a modified lysine residue bearing an isotopic tag or isobaric tag
VLEDRPLSDKTHIALDGR

SEQ ID NO: 20 - protein, 24 aa, Homo sapiens
  VARIANT (2, 12): Corresponding synthetic peptide may have modified lysine residues bearing an isotopic tag or isobaric tag
AKWDTANNPLYKEATSTFTNITYR

SEQ ID NO: 21 - protein, 22 aa, Homo sapiens
  VARIANT (6)..(6): Corresponding synthetic peptide may have a modified lysine residue bearing an isotopic tag or isobaric tag
VCDLLKAEHPTWGDEQLFQTTR

SEQ ID NO: 22 - protein, 14 aa, Homo sapiens
VPDASQDDGPAVER

SEQ ID NO: 23 - protein, 18 aa, Homo sapiens
VPDASQDDGPAVERSTEL

SEQ ID NO: 24 - protein, 12 aa, Homo sapiens
WFWEFVNATFIR

SEQ ID NO: 25 - protein, 8 aa, Homo sapiens
LQPFNEYR

SEQ ID NO: 26 - protein, 11 aa, Homo sapiens
LDIDSPPITAR

SEQ ID NO: 27 - protein, 19 aa, Homo sapiens
  VARIANT (16)..(16): Corresponding synthetic peptide may have a modified lysine residue bearing an isotopic tag or isobaric tag
LNFSHGTHEYHAETIKNVR

SEQ ID NO: 28 - protein, 22 aa, Homo sapiens
  VARIANT (8)..(8): Corresponding synthetic peptide may have a modified lysine residue bearing an isotopic tag or isobaric tag
GIFPVLCKDPVQEAWAEDVDLR

SEQ ID NO: 29 - protein, 28 aa, Homo sapiens
  VARIANT (23)..(23): Corresponding synthetic peptide may have a modified lysine residue bearing an isotopic tag or isobaric tag
TATESFASDPILYRPVAVALDTKGPEIR

SEQ ID NO: 30 - protein, 16 aa, Homo sapiens
EAEAAIYHIQLFEELR

SEQ ID NO: 31 - protein, 11 aa, Homo sapiens
  VARIANT (2)..(2): Corresponding synthetic peptide may have a modified lysine residue bearing an isotopic tag or isobaric tag
LKLLLPWLEAR

SEQ ID NO: 32 - protein, 17 aa, Homo sapiens
  VARIANT (1)..(1): Corresponding synthetic peptide may have a modified lysine residue bearing an isotopic tag or isobaric tag
KDPELWGSVLLESNPYR

SEQ ID NO: 33 - protein, 15 aa, Homo sapiens
  VARIANT (12)..(12): Corresponding synthetic peptide may have a modified lysine residue bearing an isotopic tag or isobaric tag
NLQNLLILTAIKADR

SEQ ID NO: 34 - protein, 20 aa, Homo sapiens
  VARIANT (1)..(1): Corresponding synthetic peptide may have a modified lysine residue bearing an isotopic tag or isobaric tag
KFDLGQDVIDFTGHALALYR

SEQ ID NO: 35 - protein, 22 aa, Homo sapiens
  VARIANT (3)..(3): Corresponding synthetic peptide may have a modified lysine residue bearing an isotopic tag or isobaric tag
YGKSPYLYPLYGLGELPQGFAR

SEQ ID NO: 36 - protein, 20 aa, Homo sapiens
  VARIANT (19)..(19): Corresponding synthetic peptide may have a modified lysine residue bearing an isotopic tag or isobaric tag
NPYYGGESSSITPLEELYKR

SEQ ID NO: 37 - protein, 11 aa, Homo sapiens
  VARIANT (1)..(1): Corresponding synthetic peptide may have a modified lysine residue bearing an isotopic tag or isobaric tag
KQNDVFGEAEQ

SEQ ID NO: 38 - protein, 10 aa, Homo sapiens
WDTANNPLYK

SEQ ID NO: 39 - protein, 12 aa, Homo sapiens
EATSTFTNITYR

SEQ ID NO: 40 - protein, 23 aa, Homo sapiens
DTANNPLYKEATSTFTNITYRGT

SEQ ID NO: 41 - protein, 15 aa, Homo sapiens
DTVRDGAGRFLKSLV

SEQ ID NO: 42 - protein, 17 aa, Homo sapiens
DTHYAQSLRKWNQPVSI

SEQ ID NO: 43 - protein, 10 aa, Homo sapiens
DASHLLVFTT

SEQ ID NO: 44 - protein, 11 aa, Homo sapiens
DGRLAGIVQPN

SEQ ID NO: 45 - protein, 784 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTHYAQSLRKWNQPVSIDG

SEQ ID NO: 46 - protein, 447 aa, Homo sapiens [sequence omitted]

SEQ ID NO: 47 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 48 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 49 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 50 - protein, 1675 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGQPQLMLTAGPSVAVPPQAPFGYGYTAPPYGQPQPGFGYSM

SEQ ID NO: 51 - protein, 1639 aa, Homo sapiens
MAQILPIRFQEHLQLQNLGINPANIGFSTLTMESDKFICIREKVGEQAQVVIIDMNDPSNPIRRPISADSAIMNPASKVIALKAGKTLQIFNIEMKSKMKAHTMTDDVTFWKWISLNTVALVTDNAVYHWSMEGESQPVKMFDRHSSLAGCQIINYRTDAKQKWLLLTGISAQQNRVVGAMQLYSVDRKVSQPIEGHAASFAQFKMEGNAEESTLFCFAVRGQAGGKLHIIEVGTPPTGNQPFPKKAVDVFFPPEAQNDFPVAMQISEKHDVVFLITKYGYIHLYDLETGTCIYMNRISGETIFVTAPHEATAGIIGVNRKGQVLSVCVEEENIIPYITNVLQNPDLALRMAVRNNLAGAEELFARKFNALFAQGNYSEAAKVAANAPKGILRTPDTIRRFQSVPAQPGQTSPLLQYFGILLDQGQLNKYESLELCRPVLQQGRKQLLEKWLKEDKLECSEELGDLVKSVDPTLALSVYLRANVPNKVIQCFAETGQVQKIVLYAKKVGYTPDWIFLLRNVMRISPDQGQQFAQMLVQDEEPLADITQIVDVFMEYNLIQQCTAFLLDALKNNRPSEGPLQTRLLEMNLMHAPQVADAILGNQMFTHYDRAHIAQLCEKAGLLQRALEHFTDLYDIKRAVVHTHLLNPEWLVNYFGSLSVEDSLECLRAMLSANIRQNLQICVQVASKYHEQLSTQSLIELFESFKSFEGLFYFLGSIVNFSQDPDVHFKYIQAACKTGQIKEVERICRESNCYDPERVKNFLKEAKLTDQLPLIIVCDRFDFVHDLVLYLYRNNLQKYIEIYVQKVNPSRLPVVIGGLLDVDCSEDVIKNLILVVRGQFSTDELVAEVEKRNRLKLLLPWLEARIHEGCEEPATHNALAKIYIDSNNNPERFLRENPYYDSRVVGKYCEKRDPHLACVAYERGQCDLELINVCNENSLFKSLSRYLVRRKDPELWGSVLLESNPYRRPLIDQVVQTALSETQDPEEVSVTVKAFMTADLPNELIELLEKIVLDNSVFSEHRNLQNLLILTAIKADRTRVMEYINRLDNYDAPDIANIAISNELFEEAFAIFRKFDVNTSAVQVLIEHIGNLDRAYEFAERCNEPAVWSQLAKAQLQKGMVKEAIDSYIKADDPSSYMEVVQAANTSGNWEELVKYLQMARKKARESYVETELIFALAKTNRLAELEEFINGPNNAHIQQVGDRCYDEKMYDAAKLLYNNVSNFGRLASTLVHLGEYQAAVDGARKANSTRTWKEVCFACVDGKEFRLAQMCGLHIVVHADELEELINYYQDRGYFEELITMLEAALGLERAHMGMFTELAILYSKFKPQKMREHLELFWSRVNIPKVLRAAEQAHLWAELVFLYDKYEEYDNAIITMMNHPTDAWKEGQFKDIITKVANVELYYRAIQFYLEFKPLLLNDLLMVLSPRLDHTRAVNYFSKVKQLPLVKPYLRSVQNHNNKSVNESLNNLFITEEDYQALRTSIDAYDNFDNISLAQRLEKHELIEFRRIAAYLFKGNNRWKQSVELCKKDSLYKDAMQYASESKDTELAEELLQWFLQEEKRECFGACLFTCYDLLRPDVVLETAWRHNIMDFAMPYFIQVMKEYLTKVDKLDASESLRKEEEQATETQPIVYGNLSL

SEQ ID NO: 52 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 53 - protein, 599 aa, Homo sapiens
MSRSLLLWFLLFLLLLPPLPVLLADPGAPTPVNPCCYYPCQHQGICVRFGLDRYQCDCTRTGYSGPNCTIPGLWTWLRNSLRPSPSFTHFLLTHGRWFWEFVNATFIREMLMRLVLTVRSNLIPSPPTYNSAHDYISWESFSNVSYYTRILPSVPKDCPTPMGTKGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKVGSQEYSYEQFLFNTSMLVDYGVEALVDAFSRQIAGRIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTEL

SEQ ID NO: 54 - protein, 562 aa, Homo sapiens
MSRSLLLWFLLFLLLLPPLPVLLADPGAPTPVNPCCYYPCQHQGICVRFGLDRYQCDCTRTGYSGPNCTIPGLWTWLRNSLRPSPSFTHFLLTHGRWFWEFVNATFIREMLMRLVLTVRSNLIPSPPTYNSAHDYISWESFSNVSYYTRILPSVPKDCPTPMGTKGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTEL

SEQ ID NO: 55 - protein, 2541 aa, Homo sapiens
MVALSLKISIGNVVKTMQFEPSTMVYDACRIIRERIPEAPAGPPSDFGLFLSDDDPKKGIWLEAGKALDYYMLRNGDTMEYRKKQRPLKIRMLDGTVKTIMVDDSKTVTDMLMTICARIGITNHDEYSLVRELMEEKKEEITGTLRKDKTLLRDEKKMEKLKQKLHTDDELNWLDHGRTLREQGVEEHETLLLRRKFFYSDQNVDSRDPVQLNLLYVQARDDILNGSHPVSFDKACEFAGFQCQIQFGPHNEQKHKAGFLDLKDFLPKEYVKQKGERKIFQAHKNCGQMSEIEAKVRYVKLARSLKTYGVSFFLVKEKMKGKNKLVPRLLGITKECVMRVDEKTKEVIQEWNLTNIKRWAASPKSFTLDFGDYQDGYYSVQTTEGEQIAQLIAGYIDIILKKKKSKDHFGLEGDEESTMLEDSVSPKKSTVLQQQYNRVGKVEHGSVALPAIMRSGASGPENFQVGSMPPAQQQITSGQMHRGHMPPLTSAQQALTGTINSSMQAVQAAQATLDDFDTLPPLGQDAASKAWRKNKMDESKHEIHSQVDAITAGTASVVNLTAGDPAETDYTAVGCAVTTISSNLTEMSRGVKLLAALLEDEGGSGRPLLQAAKGLAGAVSELLRSAQPASAEPRQNLLQAAGNVGQASGELLQQIGESDTDPHFQDALMQLAKAVASAAAALVLKAKSVAQRTEDSGLQTQVIAAATQCALSTSQLVACTKVVAPTISSPVCQEQLVEAGRLVAKAVEGCVSASQAATEDGQLLRGVGAAATAVTQALNELLQHVKAHATGAGPAGRYDQATDTILTVTENIFSSMGDAGEMVRQARILAQATSDLVNAIKADAEGESDLENSRKLLSAAKILADATAKMVEAAKGAAAHPDSEEQQQRLREAAEGLRMATNAAAQNAIKKKLVQRLEHAAKQAAASATQTIAAAQHAASTPKASAGPQPLLVQSCKAVAEQIPLLVQGVRGSQAQPDSPSAQLALIAASQSFLQPGGKMVAAAKASVPTIQDQASAMQLSQCAKNLGTALAELRTAAQKAQEACGPLEMDSALSVVQNLEKDLQEVKAAARDGKLKPLPGETMEKCTQDLGNSTKAVSSAIAQLLGEVAQGNENYAGIAARDVAGGLRSLAQAARGVAALTSDPAVQAIVLDTASDVLDKASSLIEEAKKAAGHPGDPESQQRLAQVAKAVTQALNRCVSCLPGQRDVDNALRAVGDASKRLLSDSLPPSTGTFQEAQSRLNEAAAGLNQAATELVQASRGTPQDLARASGRFGQDFSTFLEAGVEMAGQAPSQEDRAQVVSNLKGISMSSSKLLLAAKALSTDPAAPNLKSQLAAAARAVTDSINQLITMCTQQAPGQKECDNALRELETVRELLENPVQPINDMSYFGCLDSVMENSKVLGEAMTGISQNAKNGNLPEFGDAISTASKALCGFTEAAAQAAYLVGVSDPNSQAGQQGLVEPTQFARANQAIQMACQSLGEPGCTQAQVLSAATIVAKHTSALCNSCRLASARTTNPTAKRQFVQSAKEVANSTANLVKTIKALDGAFTEENRAQCRAATAPLLEAVDNLSAFASNPEFSSIPAQISPEGRAAMEPIVISAKTMLESAGGLIQTARALAVNPRDPPSWSVLAGHSRTVSDSIKKLITSMRDKAPGQLECETAIAALNSCLRDLDQASLAAVSQQLAPREGISQEALHTQMLTAVQEISHLIEPLANAARAEASQLGHKVSQMAQYFEPLTLAAVGAASKTLSHPQQMALLDQTKTLAESALQLLYTAKEAGGNPKQAAHTQEALEEAVQMMTEAVEDLTTTLNEAASAAGVVGGMVDSITQAINQLDEGPMGEPEGSFVDYQTTMVRTAKAIAVTVQEMVTKSNTSPEELGPLANQLTSDYGRLASEAKPAAVAAENEEIGSHIKHRVQELGHGCAALVTKAGALQCSPSDAYTKKELIECARRVSEKVSHVLAALQAGNRGTQACITAASAVSGIIADLDTTIMFATAGTLNREGTETFADHREGILKTAKVLVEDTKVLVQNAAGSQEKLAQAAQSSVATITRLADVVKLGAASLGAEDPETQVVLINAVKDVAKALGDLISATKAAAGKVGDDPAVWQLKNSAKVMVTNVTSLLKTVKAVEDEATKGTRALEATTEHIRQELAVFCSPEPPAKTSTPEDFIRMTKGITMATAKAVAAGNSCRQEDVIATANLSRRAIADMLRACKEAAYHPEVAPDVRLRALHYGRECANGYLELLDHVLLTLQKPSPELKQQLTGHSKRVAGSVTELIQAAEAMKGTEWVDPEDPTVIAENELLGAAAAIEAAAKKLEQLKPRAKPKEADESLNFEEQILEAAKSIAAATSALVKAASAAQRELVAQGKVGAIPANALDDGQWSQGLISAARMVAAATNNLCEAANAAVQGHASQEKLISSAKQVAASTAQLLVACKVKADQDSEAMKRLQAAGNAVKRASDNLVKAAQKAAAFEEQENETVVVKEKMVGGIAQIIAAQEEMLRKERELEEARKKLAQIRQQQYKFLPSELRDEH

SEQ ID NO: 56 - protein, 1960 aa, Homo sapiens
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKERYYSGLIYTYSGLFCVVINPYKNLPIYSEEIVEMYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEEQIILEDQNCKLAKEKKLLEDRIAEFTTNLTEEEEKSKSLAKLKNKHEAMITDLEERLRREEKQRQELEKTRRKLEGDSTDLSDQIAELQAQIAELKMQLAKKEEELQAALARVEEEAAQKNMALKKIRELESQISELQEDLESERASRNKAEKQKRDLGEELEALKTELEDTLDSTAAQQELRSKREQEVNILKKTLEEEAKTHEAQIQEMRQKHSQAVEELAEQLEQTKRVKANLEKAKQTLENERGELANEVKVLLQGKGDSEHKRKKVEAQLQELQVKFNEGERVRTELADKVTKLQVELDNVTGLLSQSDSKSSKLTKDFSALESQLQDTQELLQEENRQKLSLSTKLKQVEDEKNSFREQLEEEEEAKHNLEKQIATLHAQVADMKKKMEDSVGCLETAEEVKRKLQKDLEGLSQRHEEKVAAYDKLEKTKTRLQQELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 57 - protein, 1382 aa, Homo sapiens
MYKGKKRHEMPPHIYAITDTAYRSMMQDREDQSILCTGESGAGKTENTKKVIQYLAYVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVMQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEELDDLLVDLDHQRQSACNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDERKQRSMAVAARKKLEMDLKDLEAHIDSANKNRDEAIKQLRKLQAQMKDCMRELDDTRASREEILAQAKENEKKLKSMEAEMIQLQEELAAAERAKRQAQQERDELADEIANSSGKGALALEEKRRLEARIAQLEEELEEEQGNTELINDRLKKANLQIDQINTDLNLERSHAQKNENARQQLERQNKELKVKLQEMEGTVKSKYKASITALEAKIAQLEEQLDNETKERQAACKQVRRTEKKLKDVLLQVDDERRNAEQYKDQADKASTRLKQLKRQLEEAEEEAQRANASRRKLQRELEDATETADAMNREVSSLKNKLRRGDLPFVVPRRMARKGAGDGSDEEVDGKADGAEAKPAE

SEQ ID NO: 58 - protein, 1134 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 59 - protein, 1066 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 60 - protein, 222 aa, Homo sapiens
MPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKVRVLSGEISKIPNSPWLGVLIGTCLILYLVIFVA

SEQ ID NO: 61 - protein, 788 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 62 - protein, 780 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTVRDGAGRFLKSLV

SEQ ID NO: 63 - protein, 14 aa, Homo sapiens
VDQLTAQLADLAAR

SEQ ID NO: 64 - protein, 22 aa, Homo sapiens
  VARIANT (14)..(14): Corresponding synthetic peptide may have a modified lysine residue bearing an isotopic tag or isobaric tag
NDASHLLVFTTDAKTHIALDGR

SEQ ID NO: 65 - protein, 16 aa, Homo sapiens
QATKDAGTIAGLNVMR

SEQ ID NO: 66 - protein, 19 aa, Homo sapiens
CDLCQEVLADIGFVKNAGR

SEQ ID NO: 67 - protein, 16 aa, Homo sapiens
TGDGKILYSQCGDVMR

SEQ ID NO: 68 - protein, 21 aa, Homo sapiens
VAPEEHPVLLTEAPLNPKANR

SEQ ID NO: 69 - protein, 20 aa, Homo sapiens
AKQIVWNGPVGVFEWEAFAR

SEQ ID NO: 70 - protein, 22 aa, Homo sapiens
KDLYANTVLSGGTTMYPGIADR

SEQ ID NO: 71 - protein, 21 aa, Homo sapiens
QLFHPEQLITGKEDAANNYAR

SEQ ID NO: 72 - protein, 23 aa, Homo sapiens
LIGDAAKNQVALNPQNTVFDAKR

SEQ ID NO: 73 - protein, 13 aa, Homo sapiens
DLTDYLMKILTER

SEQ ID NO: 74 - protein, 16 aa, Homo sapiens
HAHGDQYKATDFVADR

SEQ ID NO: 75 - protein, 18 aa, Homo sapiens
IAKGGVNDNFQGVLQNVR

SEQ ID NO: 76 - protein, 24 aa, Homo sapiens
LVAKAVEGCVSASQAATEDGQLLR

SEQ ID NO: 77 - protein, 18 aa, Homo sapiens
SKFALITWIGENVSGLQR

SEQ ID NO: 78 - protein, 15 aa, Homo sapiens
FKLEESYTLNSDLAR

SEQ ID NO: 79 - protein, 11 aa, Homo sapiens
GECEDMLSKCR

SEQ ID NO: 80 - protein, 25 aa, Homo sapiens
VLEDRPLSDKGSGDSSQVTQVSPQR

SEQ ID NO: 81 - protein, 22 aa, Homo sapiens
IIGLDQVAGMSETALPGAFKTR

SEQ ID NO: 82 - protein, 27 aa, Homo sapiens
ILAQATSDLVNAIKADAEGESDLENSR

SEQ ID NO: 83 - protein, 18 aa, Homo sapiens
IAKGGVNDNFQGVLQNVR

SEQ ID NO: 84 - protein, 15 aa, Homo sapiens
KIQALQQQADEAEDR

SEQ ID NO: 85 - protein, 24 aa, Homo sapiens
LVAKAVEGCVSASQAATEDGQLLR

SEQ ID NO: 86 - protein, 13 aa, Homo sapiens
ISKLEQQMAKIAA

SEQ ID NO: 87 - protein, 19 aa, Homo sapiens
AKFYPEDVSEELIQDITQR

SEQ ID NO: 88 - protein, 14 aa, Homo sapiens
LEVNLQAMKAQFER

SEQ ID NO: 89 - protein, 20 aa, Homo sapiens
ASLSKLGDVYVNDAFGTAHR

SEQ ID NO: 90 - protein, 25 aa, Homo sapiens
KFFYSDQNVDSRDPVQLNLLYVQAR

SEQ ID NO: 91 - protein, 19 aa, Homo sapiens
LLGWIQNKLPQLPITNFSR

SEQ ID NO: 92 - protein, 8 aa, Homo sapiens
EKIETELR

SEQ ID NO: 93 - protein, 9 aa, Homo sapiens
ITITNDQNR

SEQ ID NO: 94 - protein, 14 aa, Homo sapiens
ITPSYVAFTPEGER

SEQ ID NO: 95 - protein, 15 aa, Homo sapiens
NQLTSNPENTVFDAK

SEQ ID NO: 96 - protein, 8 aa, Homo sapiens
APIIAVTR

SEQ ID NO: 97 - protein, 10 aa, Homo sapiens
GSGTAEVELK

SEQ ID NO: 98 - protein, 17 aa, Homo sapiens
GVNLPGAAVDLPAVSEK

SEQ ID NO: 99 - protein, 9 aa, Homo sapiens
VVEGSFVYK

SEQ ID NO: 100 - protein, 14 aa, Homo sapiens
FLVFVANFDENDPK

SEQ ID NO: 101 - protein, 8 aa, Homo sapiens
THIALDGR

SEQ ID NO: 102 - protein, 10 aa, Homo sapiens
VLEDRPLSDK

SEQ ID NO: 103 - protein, 12 aa, Homo sapiens
IGDTVSFSIEAK

SEQ ID NO: 104 - protein, 8 aa, Homo sapiens
LQPFNEYR

SEQ ID NO: 105 - protein, 10 aa, Homo sapiens
QLPDAQLLAR

SEQ ID NO: 106 - protein, 18 aa, Homo sapiens
ALGHGVDLGHIYGDNLER

(19) United States
(12) Patent Application Publication
(10) Pub. No.: US 2014/0220694 A1
(43) Pub. Date: Aug. 7, 2014

(54) METHODS AND COMPOSITIONS RELATING TO PLATELET SENSITIVITY

(71) Applicants: Timothy William Goodman, Salisbury (GB); Albert Ferro, London (GB); Emma Schofield, London (GB); Malcolm Andrew Ward, Bedfordshire (GB); Christopher Floyd, London (GB)

(72) Inventors: Timothy William Goodman, Salisbury (GB); Albert Ferro, London (GB); Emma Schofield, London (GB); Malcolm Andrew Ward, Bedfordshire (GB); Christopher Floyd, London (GB)

(73) Assignees: ELECTROPHORETICS LIMITED, Surrey (GB); KING'S COLLEGE LONDON, London (GB)

(21) Appl. No.: 14/116,846

(22) PCT Filed: May 11, 2012
(86) PCT No.: PCT/GB2012/000433

§ 371 Date: Feb. 25, 2014

Related U.S. Application Data
(60) Provisional application No. 61/485,818, filed on May 13, 2011.

(51) Int. Cl. G01N 33/50

(52) U.S. Cl. CPC G01N 33/5023
USPC 436/63

(57) ABSTRACT

The invention identifies and describes proteins that are differentially expressed in platelets resistant to anti-platelet agents such as aspirin (acetylsalicyclic acid) compared to those platelets that are sensitive to such agents. Methods for determining further such differentially expressed proteins and methods for determining an individual's sensitivity to anti-platelet agents, such as aspirin, prior to administration.

### FIELD OF THE INVENTION

The invention relates to methods and compositions for determining platelet sensitivity. Specifically, but not exclusively, the invention identifies and describes proteins that are differentially expressed in platelets resistant to anti-platelet agents, e.g. aspirin (acetylsalicyclic acid) compared to those platelets that are sensitive to such agents. The invention further provides methods for determining further such differentially expressed proteins which may provide important molecular markers or targets for anti-platelet agents. Still further, the invention provides methods for determining an individual's sensitivity to anti-platelet agents, such as aspirin, prior to administration.

### BACKGROUND OF THE INVENTION

Platelet aggregation is a major cause of arterial thrombotic disorders including myocardial infarction (heart attack), stroke, and other occlusive arterial diseases. In such cases the thrombus is initiated by activation of the platelet aggregation pathway. An early step in this pathway is the conversion of arachidonic acid to prostaglandins G1/G2 by the cyclooxygenase (COX)-1 enzyme. Inhibition of COX-1 activity by aspirin is therefore widely used clinically for prophylaxis against thrombotic disease, in patients at high risk of this.

Whilst aspirin treatment of such patients has proved highly successful and economical, there remain a significant number of patients for whom the treatment is either partially or completely unsuccessful, as judged by the occurrence of recurrent arterial thrombotic events despite being on aspirin. At present there are few objective tests which are predictive of an individual's true response to anti-platelet medications (including aspirin). Estimates of the incidence of platelet resistance to aspirin treatment range from 5-75% (Kranzhofer & Ruef, 2006. Platelets 17(3): 163-169), this wide range being largely explained by the fact that there is no gold-standard definition of aspirin resistance by either biochemical or functional assays, coupled with the wide range of tests employed in such studies in attempting to define it. There are currently several published methods for assessing platelet function and response to aspirin. Whilst these tests provide a degree of objective assessment of aspirin responsiveness, none is adequately standardised for routine use in a clinical setting (Haubelt et al. 2005. Seminars in Thrombosis and Hemostasis 31(4): 404-410). Similar considerations apply to other anti-platelet medications working through different pathways, for example clopidogrel, which inhibits platelet function by blocking the P2Y12 purinergic receptor with no effect on the COX-1 pathway.

It remains commonplace for patients with clinically defined risk factors and/or a recent history of arterial thrombosis to be placed onto aspirin treatment without prior assessment of their platelet function and aspirin responsiveness. Whilst aspirin treatment is inexpensive and non-invasive, it is not without significant risk, one of the most important adverse effects being peptic ulceration with or without resultant gastrointestinal haemorrhage.

Aspirin is used in both primary and secondary prevention of atherothrombotic cardiovascular disease, due to its anti-platelet effect. It is by far the most widely used anti-platelet drug for this purpose, due to its low cost and long experience with its use, coupled with robust outcome evidence in a number of cardiovascular trials. Whilst aspirin can provide highly efficacious inhibition of platelet aggregation, a significant number of individuals have resistance to its anti-platelet effect, rendering treatment ineffective at best and at worst may lead to severe side-effects including gastric damage.

To address the issue of aspirin resistance, a number of platelet function tests have been applied. However, currently available tests do not provide any indication as to the cause of resistance. Furthermore, whilst each test is sufficiently robust for use within a specialised testing centre in a sophisticated medical system, the results are generally not comparable with those from other centres running the same test, or from other testing systems.

### SUMMARY OF THE INVENTION

The inventors have appreciated the need for a robust method of determining platelet sensitivity to anti-platelet agents such as aspirin, so that the agent can be administered to those patients who are likely to benefit, without exposing to risk those patients who are likely to derive little or no benefit due to resistance at the platelet level.

Accordingly, and at its most general, the invention provides biomarkers associated with platelet sensitivity to anti-platelet agents, in particular aspirin, and the use of these biomarkers for the diagnosis or prognosis of platelet sensitivity. Also provided is methods for determining further biomarkers; methods of diagnosing anti-platelet agent responsiveness prior to treatment; and kits for carrying out such methods.

The invention further relates to the use of one or more biomarkers associated with platelet sensitivity as diagnostic and therapeutic targets.

The inventors have performed a comparative proteomic study of platelet extracts derived from samples either known to be resistant or known to be sensitive following 1 month's oral administration of therapeutic doses of aspirin. This study identified a number of candidate markers of platelet resistance.

Surprisingly, in platelets from aspirin-resistant subjects, aspirin treatment resulted in mostly down-regulation of peptide/protein expression as compared to the same peptides/proteins in platelets from aspirin-sensitive individuals, where expression remained broadly similar or increased under aspirin treatment. Tables 3 and 4 are provided (FIG. 6) which show the proteins that are differentially expressed. The leading protein markers (biomarkers) identified within this initial discovery experiment were:

Decreasing in Resistant Platelets after Aspirin Treatment
1. Cytoplasmic Actin-1 (SPAN P60709)
2. Clathrin Heavy Chain 1 (SPAN Q00610)
3. 78 kDa Glucose related protein (GRP-78) also known as Heat Shock Protein A 5 (SPAN P11021)
4. Pyruvate kinase isozymes M1/M2 (SPAN P14618)
5. RAB GDP dissociation inhibitor alpha (SPAN P31150)
   Increasing in Resistant Platelets after Aspirin Treatment
6. Integrin beta 3 (also known as glycoprotein IIIa and CD61) (Swiss Prot Accession No (SPANP05106); (SPAN P23219).

Accordingly, the invention relates to the determination (e.g. for the purpose of diagnosis) of platelet resistance to anti-platelet agents such as aspirin using these identified biomarkers. Use of the biomarkers includes use or detection of proteins or fragments thereof, nucleic acid encoding said proteins or complement thereof, and antibodies binding to said proteins.

Although the inventors have identified the biomarkers provided in Tables 3, 4, 5 and 6 and listed above, it will be appreciated that further biomarkers may be identified using the methods described herein and these biomarkers may be used instead of, or in conjunction with, those specifically provided herein.

The invention provides the use of the presence, absence or amount of a protein selected from those provided in Tables 3, 4, 5 and 6, or more preferably, Cytoplasmic Actin-1; Clathrin Heavy Chain 1; 78 kDa Glucose related protein (GRP-78) (also known as Heat Shock Protein A 5); Pyruvate kinase isozymes M1/M2; RAB GDP dissociation inhibitor alpha; and Integrin beta 3, preferably isoform A, or a fragment thereof, or antibodies against said protein, or nucleic acids encoding said proteins or fragments thereof, as markers for the determination of platelet resistance to an anti-platelet agent.

Also provided is the use of one or more proteins selected from those provided in Tables 3, 4, 5 and 6 or, more preferably the group consisting of Cytoplasmic Actin-1; Clathrin Heavy Chain 1; 78 kDa Glucose related protein (GRP-78) (also known as Heat Shock Protein A 5); Pyruvate kinase isozymes M1/M2; RAB GDP dissociation inhibitor alpha; and Integrin beta 3, preferably isoform A, or a fragment thereof, or a nucleic acid encoding said protein or fragment thereof, or a nucleic acid which is the complement of the nucleic acid encoding said protein or fragment thereof or an antibody to said protein or fragment thereof, in a method of determining platelet resistance to an anti-platelet agent.

Accordingly, in a first aspect, there is provided a method of determining platelet sensitivity to an anti-platelet agent, said method comprising
obtaining a platelet containing sample from a patient being treated with said anti-platelet agent;
determining protein expression levels in said sample of one or more marker proteins associated with platelet resistance;
comparing said protein expression levels with corresponding reference levels; and
determining platelet sensitivity to the anti-platelet agent based on difference in expression levels of said one or more marker proteins compared to said reference levels.

Preferably, the one or more protein markers are selected from Tables 3, 4, 5 or 6.

The corresponding reference levels, or control levels, may be provided by the determination of expression levels of the respective marker proteins in a sample from the same individual prior to treatment with said anti-platelet agent. Alternatively, the reference levels may be a set of standard reference levels previously determined from expression levels of said marker proteins in platelet samples pre and post anti-platelet agent treatment respectively from a plurality of samples or in vitro studies.

In one embodiment, the method may provide contacting a platelet containing sample obtained from an individual with an anti-platelet agent;
determining protein expression levels of one or more marker proteins associated with platelet resistance selected from Table 3, Table 4, Table 5 and Table 6;
comparing said protein expression levels with those taken from said sample prior to treatment with the anti-platelet agent; and
determining platelet sensitivity to the anti-platelet agent based on changes in expression of said one or more marker proteins.

In a further aspect of the invention, there is provided a method of determining platelet sensitivity to an anti-platelet agent, said method comprising
determining protein expression levels of one or more marker proteins associated with platelet resistance selected from Table 3, Table 4, Table 5 and Table 6 in a sample obtained from an individual being treated with an anti-platelet agent;
comparing said protein expression levels with one or more reference expression levels for the same one or more marker proteins; and
determining platelet sensitivity to the anti-platelet agent based on changes in expression levels of said one or more marker proteins compared to said corresponding reference level.

As above, the reference expression levels may be provided from a previous sample obtained from the individual prior to treatment with the anti-platelet agent. Alternatively, the reference expression levels may be an average expression level determined previously for the respective marker protein from a plurality of samples from other individuals and/or in vitro studies as representing anti-platelet agent resistance or anti-platelet sensitivity.

In one embodiment, the method comprises the steps of contacting the platelet containing sample with a solid support having immobilised thereon one or more binding agents having binding sites which are capable of specifically binding to the one or more marker proteins, antibody or nucleic acid under conditions in which the one or more marker proteins, antibody or nucleic acid bind to the binding agent; and determining the presence or amount of the one or more marker proteins, antibody or nucleic acid bound to the binding agent.

This aspect of the invention uses a platelet containing sample obtained from an individual being treated with the anti-platelet agent. This is a preferred embodiment of the invention and one that is most likely to be carried out by a clinician. However, it is also possible that determination of anti-platelet sensitivity could be carried out on a platelet containing sample obtained from an individual which is then treated in vitro with the anti-platelet agent. Changes in expression levels of the one or more marker proteins can then be determined by comparing the levels obtained from the sample pre and post anti-platelet treatment.

In some embodiments, the binding agent is an antibody or fragment thereof which is capable of binding to a marker protein or part thereof. In other embodiments, the binding agent may be a nucleic acid molecule capable of binding (i.e. complementary to) the sequence of the nucleic acid to be detected.

The method may further comprise contacting the solid support with a developing agent that is capable of binding to the occupied binding sites, unoccupied binding sites or the one or more marker proteins, antibody or nucleic acid.

The developing agent may comprise a label and the method may comprise detecting the label to obtain a value representative of the presence or amount of the one or more marker proteins, antibody or nucleic acid in the sample.

The label may be, for example, a radioactive label, a fluorophor, a phosphor, a laser dye, a chromogenic dye, a macromolecular colloidal particle, a latex bed which is coloured, magnetic or paramagnetic, an enzyme which catalyses a reaction producing a detectable result or the label is a tag.

Preferably the one or more protein markers are selected from the group consisting of Cytoplasmic Actin-1; Clathrin Heavy Chain 1; 78 kDa Glucose related protein (GRP-78) (also known as Heat Shock Protein A 5); Pyruvate kinase isozymes M1/M2; RAB GDP dissociation inhibitor alpha; and Integrin beta 3, preferably isoform A, or a fragment thereof.

In a preferred embodiment, the method uses a plurality of marker proteins or fragments thereof, e.g. two or more, three or more, four or more, five or more or six or more. Preferably, the plurality of marker proteins includes Integrin beta 3 isoform A or a fragment thereof. In one embodiment, the fragment is a C-terminal fragment of Integrin beta 3 isoform A, preferably comprising the sequence AKWDTANNPLYKEATSTFTNITYR (SEQ ID NO.1); or AKWDTANNPLYKEATSTFTNITYRGT (SEQ ID NO.2).

Many assays are known in the art for determining the presence or amounts of proteins, antibodies or nucleic acid molecules in a sample. A number of these are discussed in the detailed description below.

Accordingly, the method may comprise determining the presence or amount of a plurality of marker proteins or nucleic acids associated with resistance to anti-platelet agents in a single sample. For example, a plurality of binding agents may be immobilised at predefined locations on the solid support.

The invention also relates to a method of determining further marker proteins which may be used in proteomic analysis of platelet sensitivity to anti-platelet agents, e.g. drugs or medicaments. The method comprises the steps of obtaining a first sample of platelets that are known to be resistant to anti-platelet agents such as aspirin and obtaining a second sample of platelets that are known to be sensitive to anti-platelet agents such as aspirin. The method then includes the step of comparing the levels of proteins expressed in the first and second platelet samples and determining which proteins are differentially expressed in resistant platelets.

Further marker proteins determined by this method may be used in any of the aspects of the present invention. The invention also provides binding agents which are specific for the one or more protein markers for use in determining the presence, increase or decrease in expression of the one or more marker proteins.

The binding agent may be an antibody specific for a protein marker or a part thereof, or it may be a nucleic acid molecule which binds to a nucleic acid molecule representing the presence, increase or decrease of expression of a protein marker, e.g. a mRNA sequence.

The inventors have determined a number of protein markers (see Tables 3, 4, 5 and 6), and have identified from these a leading group comprising Cytoplasmic Actin-1; Clathrin Heavy Chain 1; 78 kDa Glucose related protein (GRP-78) (also known as Heat Shock Protein A 5); Pyruvate kinase isozymes M1/M2; RAS GDP dissociation inhibitor alpha; and integrin beta 3, including isoforms A, B and C.

The inventors have surprisingly found that the quantitative change in expression levels for integrin beta 3 was only observed for one prototypic peptide of integrin beta 3 which represented the C-terminus of the ‘A’ isoform. The inventors have found that an antibody specific for the C-terminus of integrin beta 3 as opposed to an antibody specific for the N-terminus) showed significantly elevated expression levels in post-treatment extracts of platelets from aspirin-resistant subjects whilst it remained relatively constant in platelets from aspirin-sensitive individuals.

Accordingly, in a preferred embodiment, the invention provides a binding agent which is capable of specifically binding to the C-terminus of integrin beta 3 isoform A for use in a method of the invention. This binding agent may be an antibody or part thereof.

The inventors have shown that there is a preferential enrichment of integrin beta 3 isoform A in aspirin resistant platelets. However, the inventors have also shown that there is no corresponding increase in integrin beta 3 in general. This was determined by detecting the protein via the N-terminal domain which is common to all three isoforms. Thus, by way of explanation of this determination, it seems as if an increase in isoform A is accompanied by a decrease in expression of isoform B and/or isoform C. Without wishing to be bound to any particular theory, the inventors hypothesize that differences in integrin beta 3 isoform distributions may account, at least in part, for the resistance mechanism in platelets to anti-platelet agents.

Therefore, the invention also includes binding members directed to integrin beta 3 isoforms B and/or C for use in a method of the present invention. In one embodiment, the methods of the invention may include determining changes in expression levels of integrin beta 3 isoforms A, B and C relative to each other. Thus, the method may require the use of a binding member independently specific for each of the C-terminus of integrin beta 3 isoforms A, B and C. The relative expression levels of each isoform can then be determined and compared.

In one embodiment, where the binding agent is a nucleic acid sequence, it is preferably capable of hybridising with a nucleic acid molecule comprising sequence encoding amino acid sequence selected from the group consisting of

integrin beta 3 isoform A (IGB3A)
(SEQ ID NO. 1)
AKWDTANNPLYKEATSTFTNITYR;
or
(SEQ ID NO. 2)
AKWDTANNPLYKEATSTFTNITYRGT;
integrin beta 3 isoform B (IGB3B)
(SEQ ID NO. 3)
AKWDTVRDGAGRFLKSLV;
and
Integrin beta 3 isoform C (IGB3C)
(SEQ ID NO. 4)
AKWDTHYAQSLRKWNQPV.

Where the binding member is an antibody, the antibody may be specific for any part of a protein comprising the amino acid sequences provided above.

The antibodies raised against specific marker proteins may be anti- to any biologically relevant state of the protein. Thus, for example, they can be raised against the unglycosylated form of a protein which exists in the body in a glycosylated form, against a precursor form of the protein, or a more mature form of the precursor protein, e.g. minus its signal sequence, or against a peptide carrying a relevant epitope of the marker protein.

The binding agents in accordance with the invention are preferably bound to a solid support. This may be in the form of an antibody array or a nucleic acid microarray. Arrays such as these are well known in the art. In a preferred embodiment, the binding agents of the invention contained on the array form more than 20%, 30%, 40%, 50%, 60%, 70%, 80%, or 90% of the total number of binding agents on the array.

In a preferred embodiment of the invention, the method comprises determining the expression level of one or more of the peptides selected from SEQ ID NOs 5 to 42 and SEQ ID NOs 43 and 44 in a sample obtained from an individual treated with the anti-platelet agent by Selected Reaction Monitoring using one or more of the corresponding transitions listed in Table 2 and FIGS. 7 and 8; comparing said peptide levels with peptide levels previously determined to represent platelet resistance to said anti-platelet agent; and determining platelet sensitivity to the anti-platelet agent based on changes in expression of said one or more peptides. The comparison step may include determining the amount of peptides from the treated individual with known amounts of corresponding synthetic peptides. The synthetic peptides are identical in sequence to the peptide obtained from the individual, but may be distinguished by a label such as an isobaric tag or a heavy isotope.

Alternatively, the method may comprise firstly determining the expression levels of one or more peptides selected from SEQ ID NOs 5 to 42 and SEQ ID NOs 43 and 44 in a platelet containing sample obtained from an individual by Selected Reaction Monitoring using one or more of the corresponding transitions listed in Table 2 and FIGS. 7 and 8; treating said sample with an anti-platelet agent; secondly determining the expression levels of the same one or more peptides selected from SEQ ID NOs 5 to 42 and SEQ ID NOs 43 and 44 in the treated sample by Selected Reaction Monitoring using one or more of the corresponding transitions listed in Table 2 and FIGS. 7 and 8; comparing the peptide levels between the sample obtained from the individual and the sample treated with the anti-platelet agent; and determining platelet sensitivity to the anti-platelet agent based on changes in expression of said one or more peptides.

Preferably, the one or more peptides is selected from

Myosin
SEQ ID NO. 5
LK*K*ANLQIDQINTDLNLER
SEQ ID NO. 6
EK*QLAAENR
SEQ ID NO. 7
DELADEIANSSGK*GALALEEK*R
SEQ ID NO. 8
INFDVNGYIVGANIETYLLEK*SR
Talin-1
SEQ ID NO. 9
ALEATTEHIR
SEQ ID NO. 10
DPPSWSVLAGHSR
SEQ ID NO. 11
VSEK*VSHVLAALQAGNR
SEQ ID NO. 12
LAQVAK*AVTQALNR
SEQ ID NO. 13
K*FFYSDQNVDSR
Vinculin
SEQ ID NO. 14
EAEAASIK*IR
SEQ ID NO. 15
GILSGTSDLLLTFDEAEVR
SEQ ID NO. 16
SLGEISALTSK*LADLR
SEQ ID NO. 17
DPSASPGDAGEQAIR
ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN
COX-I
SEQ ID NO. 21
VCDLLK*AEHPTWGDEQLFQTTR
SEQ ID NO. 22
VPDASQDDGPAVER
SEQ ID NO. 23
VPDASQDDGPAVERSTEL
SEQ ID NO. 24
WFWEFVNATFIR
SEQ ID NO. 25
LQPFNEYR
Pyruvate kinase
SEQ ID NO. 26
LDIDSPPITAR
SEQ ID NO. 27
LNFSHGTHEYHAETIK*NVR
SEQ ID NO. 28
GIFPVLCK*DPVQEAWAEDVDLR
SEQ ID NO. 29
TATESFASDPILYRPVAVALDTK*GPEIR
SEQ ID NO. 30
EAEAAIYHIQLFEELR
Clathrin
SEQ ID NO. 31
LK*LLLPWLEAR
SEQ ID NO. 32
K*DPELWGSVLLESNPYR
SEQ ID NO. 33
NLQNLLILTAIK*ADR
RAB GDP
SEQ ID NO. 34
K*FDLGODVIDFTGHALALYR
SEQ ID NO. 35
YGK*SPYLYPLYGLGELPQGFAR
SEQ ID NO. 36
NPYYGGESSSITPLEELYK*R
SEQ ID NO. 37
K*QNDVFGEAEQ

Symbol K* represents a modified lysine residue bearing an isotopic tag or isobaric tag.

More preferably, the peptides are selected from any one or more of the following peptides along with their respective transitions found in Table 2:—

Myosin
SEQ ID NO. 5
LK*K*ANLQIDQINTDLNLER
Talin-1
SEQ ID NO. 9
ALEATTEHIR
SEQ ID NO. 10
DPPSWSVLAGHSR
SEQ ID NO. 11
VSEK*VSHVLAALQAGNR
Vinculin
SEQ ID NO. 14
EAEAASIK*IR
SEQ ID NO. 15
GILSGTSDLLLTFDEAEVR
ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVETT
SEQ ID NO. 44
DGRLAGIVQPN
RAB GDP
SEQ ID NO. 34
K*FDLGQDVIDFTGHALALYR

Symbol K* represents a modified lysine residue bearing an isotopic tag or isobaric tag.

Even more preferably, the peptides and their respective transitions are one, two or three selected from:—

ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN

Symbol K* represents a modified lysine residue bearing an isotopic tag or isobaric tag.

A method of the invention may comprise extracting proteins from a platelet containing sample obtained from an individual. The extracted proteins may be labelled with a tag, e.g. an isotopic tag. The method may include fragmenting the protein using an enzyme (e.g. trypsin, ArgC or AspN) which digests the labelled proteins to produce a population of peptides corresponding to the peptides provided in Table 2. The method then preferably includes measuring the relative abundance of one or more of said peptides using Selected Reaction Monitoring (SRM) of one or more the relevant transitions listed in Table 2 compared to the known abundance of a control synthetic peptide. It will be understood by the skilled practitioner art that the transitions listed in Table 2 are specific for peptides when they are labelled with isotopic Tandem Mass Tags. The equivalent transitions for unlabelled peptides, or peptides bearing other labels, can be readily calculated.

The invention further provides preparations comprising one or more synthetic peptides selected from the group provide in Table 2 (SEQ ID NOs. 5 to 37) and SEQ ID Nos 38 to 42 and SEQ ID NOs 43 and 44.

More preferably, the preparation comprises synthetic peptides selected from

Myosin
SEQ ID NO. 5
LK*K*ANLQIDQINTDLNLER
Talin-1
SEQ ID NO. 9
ALEATTEHIR
SEQ ID NO. 10
DPPSWSVLAGHSR
SEQ ID NO. 11
VSEK*VSHVLAALQAGNR
Vinculin
SEQ ID NO. 14
EAEAASIK*IR
SEQ ID NO. 15
GILSGTSDLLLTFDEAEVR
ITGB3
SEQ ID NO. 19
K*LTSNLR
SEQ ID NO. 19
VLEDRPLSDK*THIALDGR
SEQ ID NO. 20
AK*WDTANNPLYK*EATSTFTNITYR
RAB GDP
SEQ ID NO. 34
K*FDLGQDVIDFTGHALALYR

Even more preferably, the preparation comprises one, two or three synthetic peptides selected from

ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN

One or more of these synthetic peptides may be included in a kit for carrying out the methods of the present invention. The synthetic peptides may be labelled such that they can be compared to the endogenous peptides and relative abundance can be determined.

In a further aspect of the invention, there is provided a kit for use in determining platelet sensitivity to an anti-platelet agent in an individual. The kit allows the user to determine the presence or amount of an analyte selected from one or more marker proteins or fragments thereof, one or more antibodies against said marker proteins and a nucleic acid molecule encoding said marker protein or a fragment thereof, in a sample obtained from said individual; the kit comprising
(a) a solid support having a binding agent capable of binding to the analyte immobilised thereon;
(b) a developing agent comprising a label; and
(c) one or more components selected from the group consisting of washing solutions, diluents and buffers.

The binding agent may be as described above. In particular, for detection of a marker protein or fragment thereof, the binding protein may be an antibody which is capable of binding to one or more of the marker proteins selected from the groups consisting of Cytoplasmic Actin-1; Clathrin Heavy Chain 1; 78 kDa Glucose related protein (GRP-78) (also known as Heat Shock Protein A 5); Pyruvate kinase isozymes M1/M2; RAB GDP dissociation inhibitor alpha; and Integrin beta 3 isoform A, B or C, or a fragment thereof.

For detection of a nucleic acid molecule, the binding agent may be a nucleic acid which is complementary to the sequence of the nucleic acid to be detected.

In one embodiment, the kit may provide the analyte in an assay-compatible format. As mentioned above, various assays are known in the art for determining the presence or amount of a protein, antibody or nucleic acid molecule in a sample. Various suitable assays are described below in more detail and each form embodiments of the invention.

The kit may be used in a method of determining platelet sensitivity to anti-platelet agents such as aspirin. This method may be performed as part of a general screening of multiple samples, or may be performed on a single sample obtained from the individual.

The kit may additionally provide a standard which provides a quantitative measure by which determination of an expression level of one or more marker proteins can be compared. The standard may indicate the levels of marker protein expression which indicate platelet resistance to anti-platelet agents such as aspirin.

The kit may also comprise printed instructions for performing the method.

In one embodiment, the kit for the determination of anti-platelet agent resistance or sensitivity contains a set of one or more antibody preparations capable of binding to one or more of the marker proteins, a means of incubating said antibodies with a platelet sample or extract obtained from an individual, and a means of quantitatively detecting binding of said proteins to said antibodies. The kit may also contain a set of additional reagents and buffers and a printed instruction manual detailing how to perform the method and optionally how to interpret the quantitative results as being indicative of anti-platelet agent resistance or sensitivity.

In a further embodiment, the kit may be for performance of a mass spectrometry assay and may comprise a set of reference peptides wherein each peptide in the set is uniquely representative of each of the one or more marker proteins described above and one, preferably two and more preferably three such unique peptides are used for each protein for which the kit is designed, and wherein each set of unique peptides are provided in known amounts which reflect the levels of such proteins in a standard preparation of platelets that are sensitive to the anti-platelet agent, e.g. aspirin, and platelets that are resistant to the agent. Optionally the kit may also provide protocols and reagents for the isolation and extraction of platelets from a blood sample, a purified preparation of a proteolytic enzyme such as trypsin and a detailed protocol of the method including details of the precursor mass and specific transitions to be monitored.

In a further aspect of the invention there is provided a method for determining if anti-platelet agent resistance is due to a platelet population with innate resistance to the agent (e.g. aspirin) or is instead a transient form of resistance caused by co-administered non-steroidal anti-inflammatory drug (NSAID) other than the anti-platelet agent wherein the level of one or more of the marker proteins is determined using any of the methods of the invention in a preparation of platelets from an individual receiving both the anti-platelet agent and NSAID treatment and the detected presence or levels of said one or more marker proteins is compared to levels indicative of anti-platelet agent resistance. If the detected presence or levels are consistent with anti-platelet agent resistance the patient is confirmed as having innate resistance whereas if the presence or levels of the one or more marker proteins appear to be normal then resistance is due to competitive binding of COX-1 by co-administered NSAID's or other factors such as non-adherence to therapy, poor drug adsorption and accelerated drug metabolism.

In a further embodiment, the invention provides a method of optimising anti-platelet treatment in an individual by
(a) providing a short course of anti-platelet agent treatment, e.g. administering the agent, e.g. aspirin, to the individual;
(b) measuring the level of one or more of a marker proteins selected from the group consisting of integrin beta, preferably integrin beta-3A, pyruvate kinase isoenzymes M1/M2, clathrin heavy chain, RAB GDP dissociation inhibitor alpha, prostaglandin G/H synthase 1 (COX1), actin-1 and heat shock protein A5, in a platelet containing sample obtained from said individual at the end of the anti-platelet induction phase;
(c) comparing the level of said one or more marker proteins detected in the individual's platelet sample against reference expression levels known to represent anti-platelet agent resistance; and
(d) selecting an appropriate anti-platelet therapy dependent upon the presence or absence of platelet resistance.

The methods of the invention are preferably in vitro methods carried out on a platelet containing sample obtained from an individual. The sample used in the methods is preferably a biological sample such as a blood or blood product, e.g. serum or plasma.

The sample may be treated to enrich the number of platelets and or deplete the sample of unwanted matter such as non-platelet cells or proteins. A preferred method is low-speed centrifugation to pellet larger cells, in particular erythrocytes. After centrifugation the supernatant comprising platelet rich plasma may be removed for analysis by the methods of the present invention.

The anti-platelet agent is preferably a drug or medicament that inhibits platelet aggregation. More preferably the agent is an inhibitor of the COX-1 pathway or an inhibitor of COX-1 enzyme itself. In a preferred embodiment, the anti-platelet agent is aspirin.

The materials and methods of the invention may be used to determine anti-platelet sensitivity as part of a prognostic monitoring of cardiovascular or cerebrovascular disease in an individual undergoing treatment with anti-platelet agent. The cardiovascular disease may include ischaemic heart disease e.g. stable angina, and acute coronary syndrome such as myocardial infarction. The cerebrovascular disease may be transient ischaemic attack or ischaemic stroke.

Embodiments of the present invention will now be described by way of example and not limitation with reference to the following accompanying figures. All documents mentioned herein are incorporated herein by reference.

### BRIEF DESCRIPTION OF THE FIGURES

FIG. 1 Workflow for the generation of samples for both the discovery and evaluation studies. FIG. 1A: The workflow demonstrates how the same intact labeled sample set can be used for both the TMTsixplex discovery and evaluation experiments. The discovery phases employed GeLC-MS/MS and the evaluation phase used LC-MS-SRM. Further evaluation of candidate markers for aspirin resistance was carried out by Western blotting using fresh aliquots of the unlabeled subject samples. FIG. 1B: Tandem Mass Tags®. TMTsixplex (229Da) are an isobaric set of six mass tags with five isotopic substitutions: application in discovery studies e.g. time-course, dose-response and replicate analyses.

FIG. 2 Identification of candidate proteins of aspirin resistance by monitoring changes in protein levels pre: post treatment with aspirin. FIG. 2A Functions of the platelet proteins identified in the discovery phase experiment. Total number of proteins identified was 565. FIG. 2B OPLS analysis of TMT discovery phase data. Peptides showing differentially changing levels post:pre aspirin treatment are observed.

FIG. 3

Table 1 TMT labeling strategy for the discovery phase experiment. Twelve samples in total (pre and post aspirin treatment from two aspirin resistant and four aspirin sensitive) subjects were labeled in two TMTsixplex experiments. Each TMT sixplex experiment contained samples from one aspirin resistant and two aspirin sensitive subjects.

Table 2 Summary of the peptides selected for SRM quantitation from each of the candidate proteins. The Q1 precursor m/z for each peptide are given along with the respective Q3 transitions (up to six per peptide). Peptides and SRM transitions highlighted in bold were taken forward for the quantitation of candidate proteins in the subject samples. Peptides highlighted by underlined text were removed from the final quantitation of patient samples as time-aligned SRMs were not reproducibly detected in all subject samples. Peptides highlighted in italics were not included in the final SRM method as time-aligned SRMs were not detected during the SRM method development stage.

FIG. 4 Total ion chromatogram selected peptides analysed by SRM. The final SRM method measured 64 transitions, which represented 16 peptides from 8 proteins (5 candidate proteins and 3 normalisation proteins).

FIG. 5 Analysis of ITGB3. FIG. 5a, shows the sequences of the three different isoforms of ITGB3 that only vary at the C-terminal end. FIG. 5b, Plot of post:pre ratios of integrin beta 3 peptides analysed by SRM. Peptides selected for SRM are underlined and highlighted in bold type in FIG. 5a. Peptide 1 (AKWDTANNPLYKEATSTFTNITYR—SEQ ID NO. 1) is specific to isoform A of ITGB3, whereas peptides 2˜5 represent consensus sequences for all three ITGB3 isoforms. FIG. 5c, Plot of post:pre ratios of ITGB3 peptides analysed by Western blotting using antibodies raised against the N- and C-terminus of ITGB3. The N-terminal antibody measures all three isoforms of ITGB3, whereas the C-terminal antibody is raised against a sequence that is specific to isoform A.

FIG. 6

Table 3 Top 20 peptides found to decrease in aspirin-resistant platelets compared to sensitive subjects.

Table 4 Top 20 peptides found to increase in aspirin-resistant platelets relative to sensitive subjects.

FIG. 7 Potential transitions suitable for SRM of ITGB3 isoform A in human platelets sequences WDTANNPLYK (SEQ ID NO.38) and EATSTFTNITYR (SEQ ID NO. 39).

FIG. 8 Potential transitions suitable for SRM of ITGB3 isoforms A, B and C in human platelets.

ITGB3A
SEQ ID NO. 40
DTANNPLYKEATSTFTNITYRGT
ITGB3B
SEQ ID NO. 41
DTVRDGAGRFLKSLV
ITGB3C
SEQ ID NO. 42
DTHYAQSLRKWNQPVSI

FIG. 9 FASTA sequences of protein markers.

FIG. 10

Table 5 List of peptides, transition masses and mass spectrometer settings for TSQ Vantage (Thermo Scientific) used in the Integrin Beta 3 Isotyping Assay

Table 6 List of peptides, transition masses and mass spectrometer settings for TSQ Vantage (Thermo Scientific) used in the Aspirin Resistance Biomarker SRM Assay.

Table 7 Protein expression level and assay variability for 16 peptides representing six biomarkers of aspirin resistance.

FIG. 11 Standard curves for AspN peptides representing integrin beta 3. Panel A—isoform A; Panel B—isoform B; Panel C isoform C; Panels D & E—total integrin beta 3. Results are the mean of three replicate measures performed on the same day. Error bars are plotted for all samples.

FIG. 12 Ratio of expression of Integrin beta 3 isoform A (solid bar) and total (light and shaded bars) following aspirin exposure for one month. Horizontal line shows a ratio of 1. Bars above the line are increased in response to aspirin and bars below are decreased. Samples 59 & 85 are platelets from aspirin resistant patients. Samples 6, 13, 22, 29, 30, 31, 34, 35, 39 & 99 are platelets from aspirin sensitive individuals.

FIG. 13 Standard curves for tryptic peptides representing six biomarkers of aspirin resistance. Results are the mean of three replicate measures performed on the same day. Error bars are plotted for all samples.

FIG. 14 Ratio of expression of six platelet protein biomarkers of aspirin resistance. Integrin beta 3 (total) and Integrin beta 3 isoform A, heat shock protein A5, pyruvate kinase isozyme M1/M2, RAB GDP1 alpha & prostaglandin G/H synthase. Horizontal line shows a ratio of 1. Bars above the line are increased in response to aspirin and bars below are decreased. Samples 59 & 85 are platelets from aspirin resistant patients. Samples 6, 13, 22, 29, 30, 31, 34, 35, 39 & 99 are platelets from aspirin sensitive individuals.

### DETAILED DESCRIPTION

The term “anti-platelet agent” includes an anti-platelet drug or medicament that inhibits platelet aggregation. The agent includes an inhibitor of the COX-1 pathway or an inhibitor of COX-1 enzyme itself. In a preferred embodiment, the anti-platelet agent is aspirin.

The term “antibody” includes polyclonal antiserum, monoclonal antibodies, fragments of antibodies such as single chain and Fab fragments, and genetically engineered antibodies. The antibodies may be chimeric or of a single species.

“Resistance” to anti-platelet agents, and in particular “aspirin resistance” means the characteristic of platelets to retain the capacity to aggregate in the presence of therapeutic doses/concentrations of agents e.g. aspirin, and is intended to be interpreted in its broadest context.

“Mass spectrometry assay” means any quantitative method of mass spectrometry including but not limited to selected reaction monitoring (SRM), multiple reaction monitoring (MRM), absolute quantitation using isotope-doped peptides (AQUA), Tandem Mass Tags with SRM (TMT-SRM) and TMTcalibrator.

The term “marker protein” or “biomarker” includes all biologically relevant forms of the protein identified, including post-translational modification. For example, the marker protein can be present in the platelets in a glycosylated, phosphorylated, multimeric or precursor form.

The term “control” refers to a human subject or a platelet sample therefrom wherein the platelets are sensitive to treatment by an anti-platelet agent.

“Differential expression” as used herein, refers to at least one recognisable difference in protein or nucleic acid expression, it may be a quantitatively measurable, semi-quantitatively estimatable or qualitatively detectable difference in tissue or body fluid expression. Thus, a differentially expressed protein or nucleic acid may be strongly expressed in tissue or body fluid in the first state (e.g. sensitive state) and less strongly expressed or not expressed at all in a second state (e.g. resistant state). Conversely, it may be strongly expressed in tissue or body fluid in the second state (e.g. resistant state) and less strongly expressed or not expressed at all in the first state (e.g. sensitive state). Further, expression may be considered differential if the protein or nucleic acid undergoes any recognisable change between the two states under comparison.

The terminology “increased/decreased concentration . . . compared with a control sample” does not imply that a step of comparing is actually undertaken, since in many cases it will be obvious to the skilled practitioner that the concentration is abnormally high or low. Further, the comparison made can be with the concentration previously seen in the same subject at an earlier stage of treatment or before treatment has commenced.

The term “diagnosis”, as used herein, includes determining whether platelets are resistant to an anti-platelet agent treatment. The diagnosis can serve as the basis of a prognosis as to the future outcome for the patient.

The term “sample” as used herein includes a biological sample such as a blood or blood product, e.g. serum or plasma. The sample may be treated to enrich the number or platelets and or deplete the sample of unwanted matter such as non-platelet cells or proteins.

The term “antibody array” or “antibody microarray” means an array of unique addressable elements on a continuous solid surface whereby at each unique addressable element an antibody with defined specificity for an antigen is immobilised in a manner allowing its subsequent capture of the target antigen and subsequent detection of the extent of such binding. Each unique addressable element is spaced from all other unique addressable elements on the solid surface so that the binding and detection of specific antigens does not interfere with any adjacent such unique addressable element.

The term “bead suspension array” means an aqueous suspension of one or more identifiably distinct particles whereby each particle contains coding features relating to its size and colour or fluorescent signature and to which all of the beads of a particular combination of such coding features is coated with an antibody with a defined specificity for an antigen in a manner allowing its subsequent capture of the target antigen and subsequent detection of the extent of such binding. Examples of such arrays can be found at www.luminexcorp.com where application of the xMAP® bead suspension array on the Luminex@ 100™ System is described.

The term “SPAN” means Swiss Prot Accession Number: a unique reference number relating to each specific protein in the Swiss Prot database available at http://expasy.org/sprot/. The skilled practitioner will understand that the SPAN relates to the version of Swiss Prot on 1 May 2011. Any modifications to SPANs can be tracked with the individual Swiss Prot records.

### Assays

The following describes various assays which may be carried out as a way of performing the invention. Although at its most general the invention concerns the determination of platelet sensitivity to anti-platelet agents, it is a preferred embodiment that the anti-platelet agent is aspirin. Accordingly, and for simplicity, the following text makes reference to aspirin only. However, it will be appreciated that other anti-platelet agents, especially those having the same or equivalent mechanism of action as aspirin, could be used instead of aspirin.

A preferred method of determining platelet sensitivity to aspirin comprises performing a binding assay for the one or more marker proteins. Any reasonably specific binding partner can be used. Preferably the binding partner is labelled. Preferably the assay is an immunoassay, between the marker and an antibody that recognises the protein, preferably a labelled antibody. The antibody may be raised against part or all of the marker protein. Most preferably the antibody is a monoclonal antibody or a polyclonal anti-human antiserum of high specificity for the marker protein.

Thus, the marker proteins described above are useful for the purpose of raising antibodies thereto which can be used to detect the presence, increased or decreased concentration of the marker proteins present in a diagnostic sample. Such antibodies can be raised by any of the methods well known in the immunodiagnostics field.

The sample can be taken from any valid body tissue, especially body fluid, of a mammalian or non-mammalian subject, but preferably whole blood, and most specifically a preparation of purified platelets. More preferably the subject is a mammalian species such as a mouse, rat, guinea pig, dog or primate. Most preferably the subject or individual is human.

The preferred immunoassay is carried out by measuring the extent of the protein/antibody interaction. Any known method of immunoassay may be used. A sandwich assay is preferred. In this method, a first antibody to the marker protein is bound to the solid phase such as a well of a plastic microtitre plate, and incubated with the sample and with a labelled second antibody specific to the protein to be assayed. Alternatively, an antibody capture assay can be used. Here, the test sample is allowed to bind to a solid phase, and the anti-marker protein antibody is then added and allowed to bind. After washing away unbound material, the amount of antibody bound to the solid phase is determined using a labelled second antibody, anti- to the first.

In another embodiment, a competition assay is performed between the sample and a labelled marker protein or a peptide derived therefrom. The presence of the marker protein in the sample will mean it is in competition with the labelled marker protein for a limited amount of anti-marker protein antibody bound to a solid support. The labelled marker protein or peptide thereof can be pre-incubated with the antibody on the solid phase, whereby the marker protein in the sample, if present, displaces part of the marker protein or peptide thereof bound to the antibody.

In yet another embodiment, the two antigens (said first antigen being present in the sample under test and the second antigen being provided by a labelled marker protein or fragment thereof) are allowed to compete in a single co-incubation with the antibody. After removal of unbound antigen from the support by washing, the amount of label attached to the support is determined and the amount of protein in the sample is measured by reference to standard titration curves established previously.

The label is preferably an enzyme. The substrate for the enzyme may be, for example, colour-forming, fluorescent or chemiluminescent.

The binding partner in the binding assay is preferably a labelled specific binding partner, but not necessarily an antibody. The binding partner will usually be labelled itself, but alternatively it may be detected by a secondary reaction in which a signal is generated, e.g. from another labelled substance.

In a preferred embodiment an amplified form of assay is provided, whereby an enhanced “signal” is produced from a relatively low level of protein to be detected. One particular form of amplified immunoassay is enhanced chemiluminescent assay. Conveniently, the antibody is labelled with e.g. horseradish peroxidase, which participates in a chemiluminescent reaction with luminol, a peroxide substrate and a compound which enhances the intensity and duration of the emitted light, typically 4-iodophenol or 4-hydroxycinnamic acid.

Another preferred form of amplified immunoassay is immuno-PCR. In this technique, the antibody is covalently linked to a molecule of arbitrary DNA comprising PCR primers, whereby the DNA with the antibody attached to it is amplified by the polymerase chain reaction. See E. R. Hendrickson et al., Nucleic Acids Research 23: 522-529 (1995). The signal is read out as before.

The use of a rapid microparticle-enhanced turbidimetric immunoassay such as the type embodied by M. Robers et al., “Development of a rapid microparticle-enhanced turbidimetric immunoassay for plasma fatty acid-binding protein, an early marker of acute myocardial infarction”, Clin. Chem. 1998; 44:1564-1567, significantly decreases the time of the assay. Thus, the full automation of any immunoassay contemplated in a widely used clinical chemistry analyser such as the COBAS™ MIRA Plus system from Hoffmann-La Roche, described by M. Robers et al. supra, or the AxSYM™ system from Abbott Laboratories, should be possible and applied for routine determination of platelet resistance.

Alternatively, the platelet containing sample under test can be subjected to two dimensional gel electrophoresis to yield a stained gel, where the increased or decreased concentration of the protein can be detected by an increased or decreased intensity of a protein-containing spot on the stained gel. This can then be compared with a corresponding control or comparative gel. The invention includes such a method, independently of the marker protein identification given above.

In yet another embodiment, the platelet containing sample can be subjected to Surface-Enhanced Laser Desorption Ionisation—Time of Flight mass spectrometry (SELDI-TOF). In this method the sample is typically a body fluid and is added to the surface of a SELDI-TOF ProteinChip prior to analysis in the SELDI-TOF mass spectrometer. General methods of SELDI-TOF analysis for human tissue samples are provided in international patent application WO01/25791.

In a yet further embodiment the diagnostic sample can be subjected to analysis by selective reaction monitoring (SRM) on either a triple quadrupole (QQQ) mass spectrometer or a quadrupole ion-trap (QTRAP) mass spectrometer. Based on the mass spectrometry profiles of the marker proteins described below single tryptic peptides with specific known mass and amino acid sequences are identified that possess good ionising characteristics. The mass spectrometer is then programmed to specifically survey for peptides of the specific mass and sequence and report their relative signal intensity. Using SRM it is possible to survey for up to 2, 5, 10, 15, 20, 25, 30, 40, 50 or 100 different marker proteins in a single LC-MS run. The intensities of the SRM transitions relating to unique peptides of the marker proteins in the diagnostic sample are compared with those found in samples from relevant control subjects.

In a further embodiment of the invention the SRM assay can be made more truly quantitative by the use of internal reference standards consisting of synthetic absolute quantification (AQUA) peptides corresponding to the SRM peptide of the marker protein wherein one or more atoms have been substituted with a stable isotope such as carbon-14 or nitrogen-15 and wherein such substitutions cause the AQUA peptide to have a defined mass difference to the native SRM peptide derived from the diagnostic sample. By comparing the relative ion intensity of the native SRM and AQUA peptides the true concentration of the parent protein in the diagnostic sample can thus be determined. General methods of absolute quantitation are provided in Gerber, Scott A, et al. “Absolute quantification of proteins and phosphoproteins from cell lysates by tandem MS” PNAS, Jun. 10, 2003. Vol 100. No 12. p 6940-6945 which is incorporated herein by reference.

### Proteomic Analysis of Aspirin Resistant and Aspirin Sensitive Platelets

To address the shortage of sensitive, robust and reliable markers of aspirin resistance the inventors undertook a comparative proteomic study of platelet extracts derived from samples known to be resistant and sensitive following 1 month's oral administration of therapeutic doses of aspirin (300 mg daily).

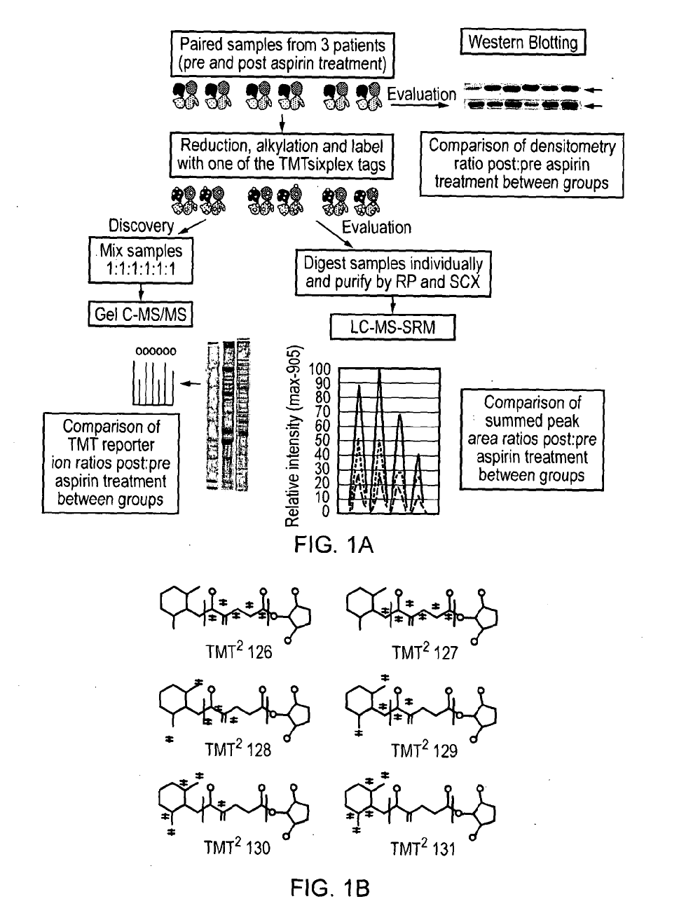

Briefly, platelets were purified from blood drawn from individuals with functionally documented aspirin resistance (n=2) or sensitivity (n=4). In each case matched samples were drawn pre- and post-aspirin treatment. Proteins were extracted, reduced and alkylated prior to labelling with a sixplex set of isobaric mass tags (TMTsixplex, Proteome Sciences plc; Refer to FIG. 1b). After labelling sets of six samples were pooled according to a randomised schedule and subjected to 1-dimensional SDS-PAGE. Each gel lane was cut into 17 bands and each band subjected to in gel digestion using trypsin prior to analysis by LC-MS/MS to allow protein identification. Relative quantitation of each protein was achieved by comparing the ion intensity and area under the curve for each of the six TMT Reporter Ions and proteins showing a difference in levels between aspirin resistant and sensitive platelets were thus identified.

A total of 565 labelled peptides were analysed and a number of candidate markers of platelet resistance identified. Surprisingly, in platelets from aspirin-resistant subjects, aspirin treatment resulted in mostly down-regulation of peptide/protein expression as compared to the same peptides/proteins in platelets from aspirin-sensitive individuals, where expression remained broadly similar or increased under aspirin treatment. The leading protein markers identified within this initial discovery experiment were:

Decreasing in Resistant Platelets after Aspirin Treatment

### 1. Cytoplasmic Actin-1 (SPAN P60709)

### 2. Clathrin Heavy Chain 1 (SPAN Q00610)

3. 78 kDa Glucose related protein (GRP-78) also known as Heat Shock Protein A 5 (SPAN P11021)
4. Pyruvate kinase isozymes M1/M2 (SPAN P14618)
5. RAB GDP dissociation inhibitor alpha (SPAN P31150)

Here, the discovery results indicate that there are multiple forms of this protein as the inventors observed some peptides which increase e.g. the C-terminal region, and others that decrease. This would suggest that changes in post translational modification status of this protein may be linked to aspirin resistance.

Increasing in Resistant Platelets after Aspirin Treatment
6. Integrin beta 3 (Swiss Prot Accession No (SPANP05106); (SPAN P23219) isoform A.

Additionally, the housekeeping proteins Talin-1 (SPAN Q97490), Myosin (SPAN P35579) and Vinculin (SPAN P18206) are included in the SRM verification method for normalisation purposes.

### Development of Mass Spectrometry Assay for Candidate Platelet-Resistance Markers

To verify the candidate markers of aspirin resistance, a mass spectrometry assay using selected reaction monitoring (SRM) of the TMT-labelled tryptic peptides prepared in the discovery experiment was developed. In mass spectrometry SRM is the scan type with the highest duty cycle and is used for monitoring one or more specific ion transition(s) at high sensitivity in a triple quadrupole (QQQ) mass spectrometer. Here, Q1 is set on the specific parent ion mass-to-charge ratio (m/z) (Q1 is not scanning) allowing a chosen peptide precursor to pass into Q2. In Q2 the peptide is fragmented and for each peptide in the SRM method the collision energy is set to produce the optimal diagnostic charged fragments (transition) of that parent ion which then pass into Q3. Finally, Q3 is sequentially set to the specific m/z of the diagnostic fragments so that only ions with this exact transition will be detected. Historically used to quantify small molecules such as drug metabolites, the same principle can be applied to peptides, either endogeneous moieties or those produced from enzymatic digestion of proteins. Again historically experiments were performed using triple quadrupole mass spectrometers but the recent introduction of hybrid instrument designs, which combine quadrupoles with ion traps, enables similar and improved experiments to be undertaken. In modern equipment such as the ABI 4000 QTRAP (hybrid ion trap/quadrupole) and TSQ Vantage (triple quadrupole) many individual SRM scans can be looped together into one experiment to detect the presence of many specific ions (up to 100 different ions) in a complex mixture. Consequently it is now feasible to measure and quantify multiple peptides from many proteins in a single chromatographic separation. The area under the SRM LC peak is used to quantitate the amount of the analyte present. In a typical quantitation experiment, a standard concentration curve is generated for the analyte of interest. When the unknown sample is then run under identical conditions, the concentration for the analyte in the unknown sample can be determined using the peak area and the standard concentration curve.

A number of candidate markers of platelet resistance were chosen based on the results of the proteomic analysis of aspirin resistant and aspirin sensitive platelets described above. The protein Prostaglandin G/H synthase 1 (COX1) was also included based on its known role as a target for aspirin and other non-steroidal anti-inflammatory medicines. Additionally, the housekeeping proteins Talin-1 (SPAN Q97490), Myosin (SPAN P35579) and Vinculin (SPAN P18206) were included in the SRM verification method for normalisation purposes.

For the present invention, a set of proteotypic peptides (peptides that are uniquely present in the target protein) were selected from all peptides detected from the aspirin resistance markers (integrin beta 3; COX1; pyruvate kinase isoenzymes M1/M2; clathrin heavy chain 1; RAS GDP dissociation inhibitor alpha) and for three housekeeping proteins (Myosin; Talin-1; Vinculin) used for assay normalisation. It was a particularly advantageous feature of the discovery method used that the same samples could be used for SRM assay development. However, it should be noted that the use of TMT labelling of intact proteins prevents trypsin cleavage at labelled lysine residues. Consequently tryptic digestion results in longer peptides with a C-terminal arginine (ARG-c like pattern). It is an aspect of the invention that smaller tryptic peptides cleaved at lysine can also be used. An example of peptides suitable for determining isoform beta 3A of integrin beta 3 (glycoprotein IIIa) are shown in FIG. 7. Similar selection of normal tryptic peptides for other aspirin resistance markers can be readily determined from the disclosed sequences herein using available computational tools such as Pinpoint™ (Thermo Scientific).

The SRM assay data confirmed the quantitative changes for integrin beta 3 seen during discovery. Integrin beta 3 has three distinct isoforms termed Beta 3A, Beta 3B and Beta 3C. FIG. 5A shows the amino acid sequence for these isoforms of human integrin beta 3 (glycoprotein IIIc) and the sites of five proteolytic peptides used to develop the quantitative SRM method. Surprisingly, the diagnostic quantitative change associated with aspirin resistance was only observed for one of the prototypic peptide of integrin beta 3 which is unique to the C-terminus of the ‘A’ isoform. The other ITGB3 tryptic peptides generated in this study were common for all three isoforms of integrin beta 3 (FIG. 5A) and did not show a difference between platelets from aspirin sensitive and aspirin resistant subjects (FIG. 5 Panels A and B).

To confirm this surprising finding, fresh extracts of platelets from the same individuals with aspirin sensitivity and aspirin resistance were subjected to western blotting using antibodies with specificity for either the N-terminus and C-terminus of ITGB3 isoform A. The intensity of staining of the integrin beta 3 band was consistent pre- and post-aspirin treatment irrespective of the platelet responsiveness status when using antibody recognising the protein N-terminus suggesting that there is no overall change in integrin beta 3 levels in platelets. However, when the C-terminal selective antibody was used, the expression of detected integrin beta 3 was significantly elevated in post-treatment extracts of platelets from aspirin-resistant subjects whilst it remained relatively constant in platelets from aspirin-sensitive individuals (FIG. 5C).

Based on the correlation between SRM and Western blot data it is reasonable to conclude that there is a preferential enrichment of ITGB3 isoform A in aspirin resistant platelets.

As stated above, the mechanism of platelet resistance to aspirin treatment is not fully understood and the inventors hypothesize that differences in ITG3B isoform distributions may account, at least in part, for the resistance mechanism.

It is therefore, a desirable embodiment of the invention to perform analysis of the ITGB3 isoform profile in platelets. This can be performed by genomic analysis of ITGB3 mRNA expression profile, analysis of ITGB3 isoform specific gene density or more preferably by measuring the Levels of the three isoforms as a percentage of total ITGB3 load. The inventors have demonstrated an ability to distinguish the Beta 3A isoform using a c-terminal peptide in an SRM assay. To generate an isoform specific SRM method the inventors have designed peptides specific for each human ITGB3 isoform based on the use of the proteolytic enzyme Asp-N which cleaves at the N-terminal side of aspartic acid residues. Specific peptides and transitions for each isoform are given in FIG. 8. These peptides each form an aspect of the present invention.

### Example 1

### Discovery of Differentially Expressed Protein Markers of Aspirin Resistance

### Determination of Aspirin Resistant Subjects

From 94 healthy subjects, 2 subjects were determined as aspirin resistance using measurements of platelet aggregommetry (agonist's arachidonic acid and ADP) and 11-dehydroTXB2 in urine. To be defined as truly aspirin resistant subjects must have an abnormal response with both measures. Subjects took 300 mg aspirin daily for 1 month.

Preparation of Platelet Samples 60 ml whole blood was obtained from subjects from the antecubital vein using a Butterfly®19-gauge needle, and collected into NaCl trisodium citrate (final concentration 0.38%). The blood was centrifuged (10 minutes, 210×g, room temperature) to produce platelet rich plasma (PRP) which was applied to a freshly washed Sepharose™ CL-2B gel column. Platelets were eluted from the column with Na-Tyrode solution (0.82% NaCl, 0.022% KCl, 0.022% KH₂PO₄, 0.1% Glucose, 0.052% HEPES (Na salt), 0.068% HEPES (acid), 0.008% MgCl₂.H₂O, 0.38& Tri-Na-Citrate) obtaining gel filtered platelets (GFP) (Fine et al., 1976, Am J Pathol 84:11-24, 197). The GFP was then divided into 5 ml volumes and centrifuged at 1660×g, 20 minutes, 4° C. to obtain a platelet pellet. The pellet was lysed with 100 μl platelet lysis buffer (0.88% NaCl, 0.21% NaF, 0.018% Na Orthovanadate, 0.39% Tris-Base), on ice for 30 minutes and sonicated on ice for a further five minutes. Lysed samples were centrifuged at 6500×g for one minute at 4° C., transferred to Eppendorfs, and stored at −80° C. until required for proteomic experiments.

### BCA Protein Assay

Platelet protein concentration was determined using a colorimetric assay with BCA (bicinchoninic acid), based on the “biuret” reaction (Smith at al., 1985, Circulation 99, 620-625). In this reaction when protein is added in alkaline solution containing Cu²⁺, reduction to Cu¹⁺ occurs and a coloured complex is formed. This complex is formed by the chelation of two molecules of BCA with one molecule of Cu¹⁺ and exhibits strong absorbance at 562 nm, which is linear with increasing protein concentrations. A 1:1 dilution of the lysate was made using ddH₂O, this process was repeated for each platelet sample. Bovine serum albumin (BSA), at concentrations 0.6, 0.9, 1.2, 1.5, 1.8, 2, 2.5, 3, 3.5, 4, 4.5, 5, 6 mg/ml were prepared as protein standards. Each BSA standard was diluted 1:1 in the same lysis buffer as was used to create the platelet lysates. A ddH₂O blank was also included, to correct all absorbance values for background level of light absorbance. Next, 10 μl of standard, blank or platelet lysate (in triplicates) were added to wells in a 96-well plate. The BCA reagent A (BCA-Na₂, 2% Na₂CO₂.H₂O, 0.16% Na₂ tartrate, 0.4% NaOH, and 0.95% NaHCO₃) and reagent B (4% CuSO₄.5H₂O in ddH₂O) were mixed in a 50:1 ratio, 200 μl was added to each well of the plate and incubated for 30 minutes, 37° C. The absorbance of each well was measured at 562 nm using a 96-well plate spectrophotometer (Spectra Max 190). Protein concentrations in the platelet lysates were determined from a standard curve of absorbance versus standard BSA concentration. The average protein platelet concentration obtained was 3.6 μg/μl

### Intact Protein Labelling of Platelet Samples

Aliquots (100 μg) of each subject sample (pre and post aspirin treatment) were individually labeled at the intact protein level with TMTsixplex (FIG. 1). All samples were labeled as per manufacturer's protocol. Pre and post aspirin treatment samples from two aspirin resistant and four aspirin sensitive subjects (12 samples in total) were selected for the discovery phase experiment. For labeling with TMTsixplex the 12 samples were labeled in two sets (Table 1). For the discovery phase experiments 30 μg aliquots of each labeled sample within a TMTsixplex experiment were combined. The remaining 70 μg was kept separate for the verification stage.

### Discovery Experiment Using GeLC-MS/MS

Prior to analysis by GeLC-MS/MS 30 μg of each sample within a TMTsixplex experiment were mixed 1:1:1:1:1:1. The combined sample was analysed by GeLC-MS/MS with the entire gel lane being excised into 15 sections. Each section was resolved over a 2 hour LC gradient (1-40% ACN; 0.05% FA, 200 nl/min) by RP-chromatographyon a 75 μM C18 PepMap column. Peptides were ionised by ESI using a Z spray source attached to a Qtof micro (Waters). Data-dependent acquisition enabled selection of precursor ions based on their intensity, for sequencing by CID fragmentation. Collision energy profiles were optimised for the analysis of TMT labeled peptides. Intact protein labeling followed by trypsin digestion produces Arg-C peptides.

For identification of platelet proteins spectral data was searched against the human IPI database using Mascot (v2.1), with fixed modification TMTsixplex (lysine) and variable modifications carbamidomethyl (C), oxidation (M). Protein identifications were validated using Scaffold 2 and manual validation. For quantitation TMT reporter ion intensities were extracted from Mascot and normalised to the sum of the total reporter ion intensity values to account for any experimental variation as a result of sample preparation. Post:pre aspirin treatment ratios for each peptide were analysed using multivariate analysis, OPLS to identify peptides that have differential levels post:pre aspirin treatment between aspirin resistant and aspirin sensitive subjects.

### Example 2

### Development of Selective Reaction Monitoring Assay for Protein Markers of Aspirin Resistance in Platelets

### Selection of Target Protein/Peptides Based on TMT Discovery Results

Peptides identified in the discovery study from candidate proteins were taken forward if they were proteotypic and had characteristics suitable for robust SRM quantitation i.e. fully hydrolysed, contained no known modifications (either in vivo or experimental) and had suitable MS/MS fragments for Q3 selection. Up to six peptides per protein were selected for targeted quantitation (Table 2).

The m/z of Q1 and Q3 transitions for selected peptides were calculated either from data-dependent sequencing in the discovery study or from in silico analysis using Pinpoint (ThermoFisher). The theoretical optimal collision energies for each transitions were also calculated using the discovery data and Pinpoint (ThermoFisher). Peptide dection and quantitation was based on time-alignment of multiple transitions per peptide (up to six transitions per peptide). Only peptides with consistant detection of multiple time-aligned transitions in all subject samples were used in the final quantitation. The final SRM method was then applied to the quantitative analysis of the experimental sample set.

### SRM Analysis

TMT-labeled subject samples (70/90 μg, check) were reduced, alkylated, digested in-solution and purified by RP and SCX chromatography. Prior to SRM analysis individual subject samples were resolved by RP-chromatography over a 9 minute ACN gradient (5˜30%) in 0.2% formic acid at 100 μl/min (20 μg total protein loaded on column). Including washes and time to equalibrate the column, the total run time of the method was 30 minutes. SRMs were visualised through Pinpoint™ software and all peak matching visually verified. Peak areas were exported into Microsoft Excel™. Transitions were summed to give a total intensity for all transitions for each peptide. The SRM peak area for each pre-aspirin sample was measured relative to the SRM peak area for each post-aspirin sample. Normalisation was achieved by comparison with housekeeping proteins, Talin and Vinculin. Ratios of all peptides relating to a particular protein were then averaged, with the exception of ITGB3 where peptides representing specific isoforms of the protein were considered separate measurements. Finally, the ratios of post:pre-aspirin treatment were compared between aspirin resistant and sensitive subjects.

### Analysis of Glycoprotein IIIa by Western Blotting

Platelet lysates from subjects (10 μg each) were mixed with equal volumes of Laemmli sample buffer (2×) (125 mM Tris-base, 4% SDS, 20% glycerol, 4% β-mercaptoethanol, 0.04% bromophenol blue), and subsequently loaded onto a 12.5% SDS-PAGE gel. Proteins were transferred onto PVDF membranes, and detected using specific antibodies as follows. Paired blots were incubated with Anti-GP111a C-terminal specific (Santa Cruz Biotechnology, Item code sc-6626, C-20) or anti GP111a N-terminal specific (Santa Cruz Biotechnology, Item code sc-6627, N-20) respectively. Bands were scanned and densitometric analysis performed using Image J system (a Java based image processing program developed by the National Institutes of Health, Bethesda, USA).

### Example 3

### SRM Assay for Integrin Beta 3 Isotyping

To provide a more objective means of measuring Integrin beta 3 and its 3 known isoforms the inventors developed a Selected Reaction Monitoring (SRM) mass spectrometry assay. Specific peptides (DASHLLVFTT (SEQ ID NO. 43) and DGRLAGIVQPN (SEQ ID NO. 44)) representing a shared region found in all isoforms of integrin beta 3 but which are otherwise unique within the human proteome were selected to give a measure of total integrin beta 3 levels. Three additional peptides corresponding to Sequence ID's 40, 41 and 42 were used to provide isotype specific quantitation for type A, B and C respectively.

To provide absolute quantitation synthetic versions of each peptide carrying a number of heavy isotopes were obtained from a commercial vendor (Thermo Scientific, Belgium).

### Isolation and Washing of Platelets

50 ml of whole blood was drawn from subjects into trisodium citrate (final concentration 0.32%) from the antecubital vein using a Butterfly® 19-guage siliconised needle. Blood was centrifuged (20 min, 200×g, room temperature (RT)) to obtain platelet rich plasma (PRP) that was acidified with the addition of 0.3M citric acid (target pH 6.5). Further centrifugation (15 min, 1200×g, RT) produced the platelet pellet that was immediately re-suspended in citrate wash buffer (refer to Appendix 1 for composition) and prostaglandin E₁ (Sigma) was added to a final concentration of 10 nM. Removal of residual red cells and monocytes was performed by further centrifugation (3 min, 200×g, RT) and decanting of the platelet rich supernatant. Final centrifugation (15 min, 1200×g, RT) produced the washed platelet pellet that was stored at −80° C.

### Preparation of Platelet Lysates

The washed platelet pellet was re-suspended in lysis buffer (NaCl 150 mM, Tris-base 32 mM, NaF 50 mM, Na orthovanadate 1 mM to which 1 ml ethylenediaminetetraacetic acid (EDTA; 0.1M stock solution) and 1 ml Triton-X 100 was added prior to adjusting to pH 7.6 with conc. HCL and volume made up to 100 ml) containing protease inhibitor cocktail (Sigma Aldrich, UK) and placed on ice for 30 min, before agitation on ice for a further 5 min. The sample was centrifuged (5 min, 9000×g, 4° C.) to remove cellular debris. The supernatant was stored at −80° C.

### Measurement of Protein Concentration

A bicinchoninic acid assay (BCA) assay was prepared for a 96-well plate spectromotometer (SpectraMAX 190, Molecular Devices) in triplicate. Bovine serum albumin (BSA) standards of concentrations 10, 8, 6, 4, 2, 1 and 0.5 mg/mL were prepared by serial dilution. BCA reagent A and B (Pierce) were prepared as 50:1 dilution and 200 μL added to each well. 10 μL of BSA standards or platelet lysates were added to their designated wells. Wells containing lysis solution provided the control. The prepared plate was then incubated (30 min, 37° C.) and the absorbance of light measured at 562 nm. A standard curve was plotted using the BSA standards and the concentration of protein within the platelet lysates was calculated from the standardised graph. Protein concentration of platelet lysates from healthy subjects was 21.34+/−5.96 ug/uL.

### Assessment of Aspirin Sensitivity

Aspirin sensitivity was assessed in individuals prescribed aspirin for secondary prevention using a combination of functional and biochemical assays. In an ischaemic heart disease population, light transmission aggregometry was performed on PRP in response to a range of agonists to assess functional platelet activity. Individuals who do not demonstrate the expected inhibition of platelet activation as a result of aspirin therapy were deemed to be functionally aspirin resistant. An ELISA was performed on whole blood to measure thromboxane A2 levels. Individuals who failed to suppress thromboxane A2 levels were deemed to be biochemically aspirin resistant.

### Integrin Beta 3 Isotyping SRM Assay

Platelet samples (up to 100 μg) were diluted 1:1 in Laemmli (2× Concentrate Sample) buffer and ran onto a Stacking gel to concentrate the entire sample into one band. Gel bands was visualised using Imperial™ Protein Stain (Pierce) and the entire band excised for in-gel digestion with AspN at the working dilution of 1:100 (Roche). Gel extracted AspN peptides were dried to completion prior to analysis using the integrin beta 3 isotyping assay.

Prior to analysis, samples were resuspended in a solution containing 5 fmol/μL of each of the heavy AQUA peptides (See table 1) and 200 μg/ml, glucagon. Prior to SRM samples were resolved by RP-chromatography over a 9 minute ACN gradient (5˜30%) in 0.2% formic acid at 100 μl/min. Integrin beta 3 isotyping assay contains 38 SRM transitions, covering 5 peptides (two peptides measuring total integrin beta and three peptides measuring each one measuring the three known isoforms A, B & C). SRM transitions are listed in Table 5 (FIG. 10). The SRM cycle time was 2 seconds with retention time windows used to maximise the scan time given to each SRM transition. Including washes and time to equalibrate the column, the total run time of the method was 30 minutes.

### Data Analysis

SRMs were visualised through Pinpoint (ThermoFisher) and all peak matching visually verified. Peak areas were exported into Microsoft Excel. Transitions were summed to give a total intensity for all transitions for each peptide. The amount of endogenous (light) peptide is calculated based on the peak area ratio relative to the 100 fmol spiked heavy peptide. For individuals where samples were available pre and post treatment with Aspirin, a post to pre ratio was calculated to assess any changes in peptide levels. The ratios of post:pre aspirin treatment were compared between aspirin resistant and sensitive subjects. For the healthy normal cohort, pre and post aspirin samples were not available and the basal pg measured peptides level per μg total protein were reported.

### Results

All peptides showed good linearity in a buffer matrix with limits of detection (LOD) and quantitation (LOQ) in the low fmol range (FIG. 11). These external standard curves were then used to quantify the amount of integrin beta 3 (total and each isotype) in platelet protein extracts. In preliminary dose-finding studies a total protein load of 10 μg was found to give integrin beta 3 levels within the mid-range of the external standard curve. In a pooled reference sample the concentration of total integrin beta 3 was calculated as being 25 pg/μg platelet protein and for isoform A the level was equivalent to 55 pg/μg platelet protein. The discrepancy between the total integrin beta 3 level and that of isoform A may reflect differential digestion efficiency at the various sites within the target protein. Overall precision and reproducibility for the measurements of total integrin beta 3 and isoform A were good with CV's for both assays being below 10%. It was not possible to detect the endogenous peptides for isoforms B & C in the platelet samples. For the isoform B peptide (Sequence ID 41) this was due to a significant matrix interference effect that could not be removed. For isoform C this was believed to be due to endogenous levels being below the limit of quantitation although a weak signal was detectable.

When the assay was applied to platelet samples from aspirin resistant or aspirin sensitive individuals (FIG. 12) the expected increase in integrin beta 3 isoform A following 1 month treatment with aspirin was seen in the two resistant patients. In the 10 aspirin sensitive patients tested the majority showed no aspirin-induced increase in isoform A levels relative to total integrin beta 3 although in some cases a small increase was observed. Significantly, in the majority of aspirin sensitive patients the level of integrin beta 3 isoform A remained level or decreased after exposure to aspirin whilst total integrin beta 3 levels remained relatively unchanged.

Finally, the assay was used to measure endogenous integrin beta 3 levels in 29 healthy individuals to assess the levels of integrin beta in a normal healthy population. Total integrin beta 3 was measured to be 6.84+/−8.6 pg/μg and integrin beta 3 isoform A at 6.797+/−2.469 pg/μg. This suggested that within a healthy population all of the integrin beta 3 found in platelets was isoform A.

### Example 2

### SRM Method for Simultaneous Measurement of Six Biomarkers of Aspirin Resistance

In addition to developing a mass spectrometry method for isotyping of integrin beta 3 in platelets the inventors have also developed a complementary method to measure six biomarkers whose expression has been shown to alter in response to aspirin treatment. The six markers are Integrin beta 3 (total) and Integrin beta 3 isoform A, heat shock protein A5, pyruvate kinase isozyme M1/M2, RAB GDP1 alpha & prostaglandin G/H synthase. For each marker two or three proteotypic tryptic peptides were selected and heavy isotope standard synthetic peptides were purchased from a commercial supplier (Thermo Scientific, Belgium).

### Materials and Methods

Platelet preparations from Example 3 were also used in the SRM method for simultaneous measurement of six biomarkers of aspirin resistance.

### Aspirin Resistance Biomarker SRM Method

Prior to analysis using Platelet Aspirin Resistance assay version 2.0 samples were resuspended in a solution containing 5 fmol/μL of each of the heavy AQUA peptides (See table 2) in the method and 200 μg/mL glucagon. Prior to SRM samples were resolved by RP-chromatography over a 9 minute ACN gradient (5-30%) in 0.2% formic acid at 100 μl/min. The Platelet Aspirin Resistance assay version 2.0 contains 96 SRM transitions, covering 17 peptides from 5 proteins. The SRM cycle time was 2 seconds with retention time windows used to maximise the scan time given to each SRM transition. SRM transitions are listed in Table 6 (FIG. 10). Including washes and time to equalibrate the column, the total run time of the method was 30 minutes.

### Results

All peptides showed good linearity in a buffer matrix with limits of detection (LOD) and quantitation (LOQ) in the low fmol range (FIG. 13). These external standard curves were then used to quantify the amount of the six biomarkers in platelet protein extracts. In preliminary dose-finding studies a total protein load of 20 μg was found to give levels within the mid-range of the external standard curve for all proteins with the exception of RAB GDP1 alpha. Assessment of increasing total protein amounts also showed a linear response in the measurement of all peptides in this assay and there was no apparent matrix interference for any peptides.

In this assay the inventors used three peptides per target protein and saw good agreement between the measured concentrations suggesting that in future only a single peptide may be needed. In the pooled standard platelet digest total integrin beta 3 was measured to be ˜3 pg/μg and integrin beta 3 isoform A at ˜2.3 pg/μg. These numbers differ from those found using the integrin beta 3 isotyping method. However, it is not intended that the levels should be comparable at this stage since they utilise different proteolytic enzymes and peptides. The results for each peptide for the six biomarkers in a pooled platelet protein digest are shown in Table 7 (FIG. 10).

When the method was applied to two aspirin resistant and 10 aspirin sensitive platelet digests the inventors saw consistent changes in aspirin-dependant expression of integrin beta 3 isoform A as well as three of the four other target proteins (heat shock protein A5, RAB GDP1 alpha & prostaglandin G/H synthase). For pyruvate kinase isozyme M1/M2 the pattern of aspirin-dependant expression was more variable (FIG. 14).

Overall these results provide a panel of platelet protein biomarkers capable of discriminating between aspirin sensitivity and resistance.

### Results and Discussion

The results presented here show discovery and verification of selected biomarker candidates for determining aspirin resistance by monitoring changes in protein expression pre- and post-aspirin treatment. Application of TMT technology enabled the same sample set to be used for both the discovery and evaluation stage. Samples were labelled at an intact protein level to reduce the technical variation often seen as a result of sample processing and digestion when labelling at the peptide level.

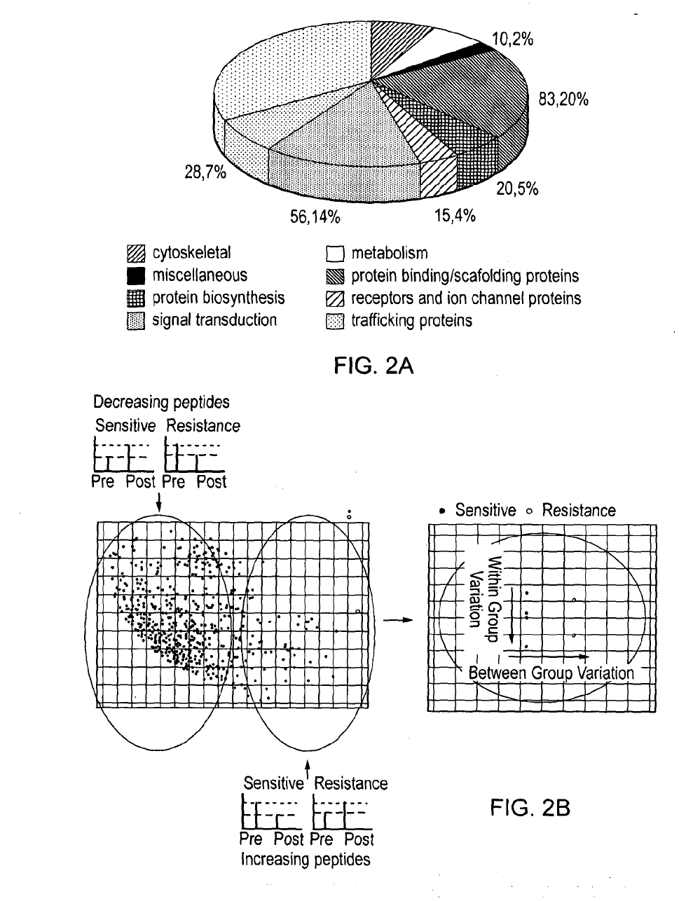

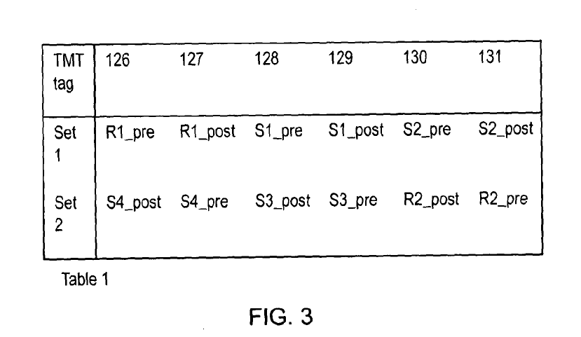

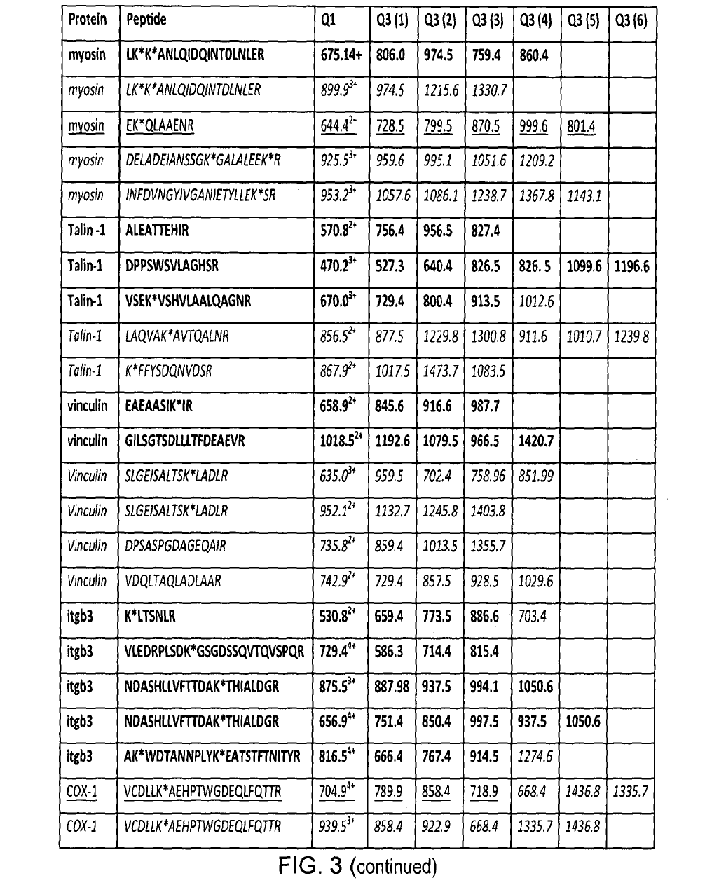

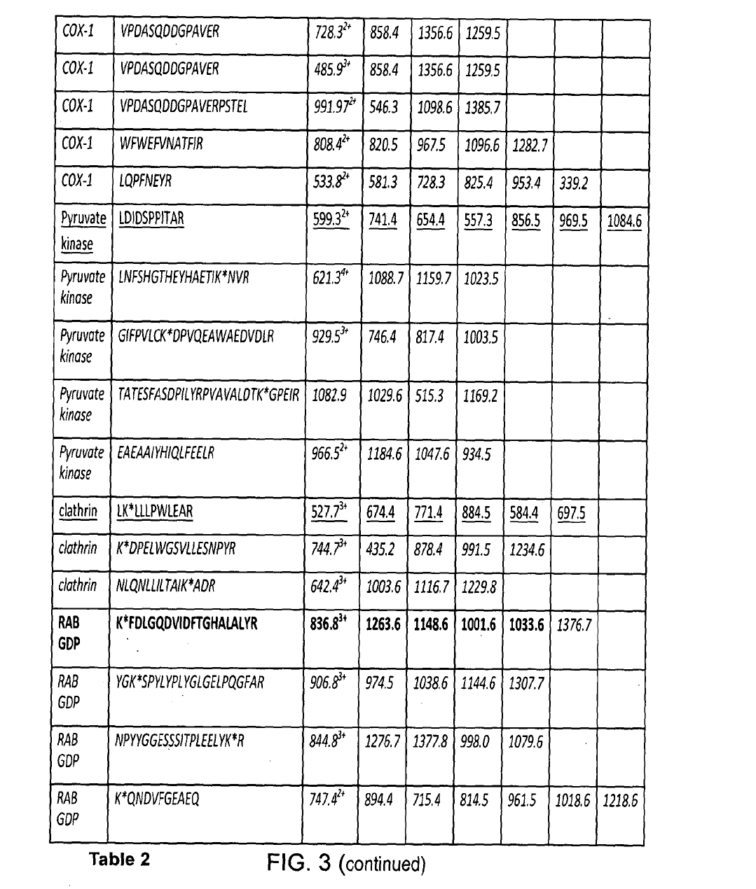

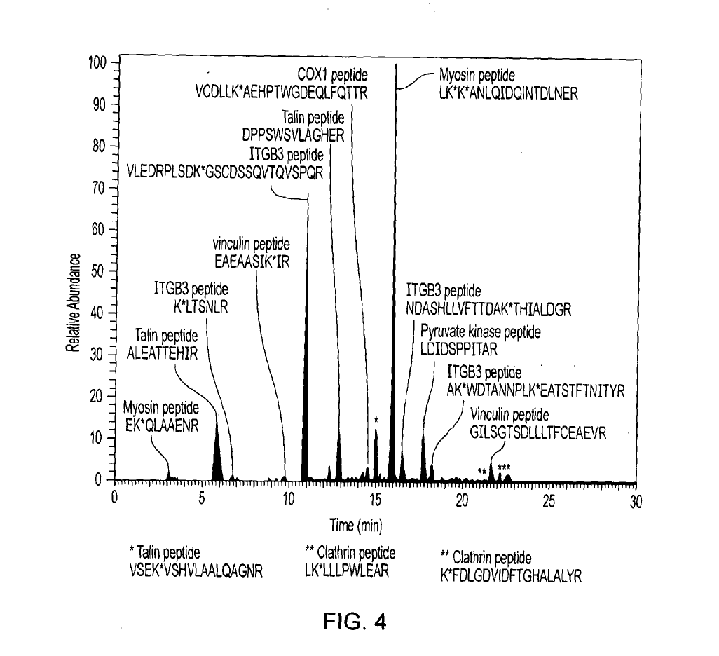

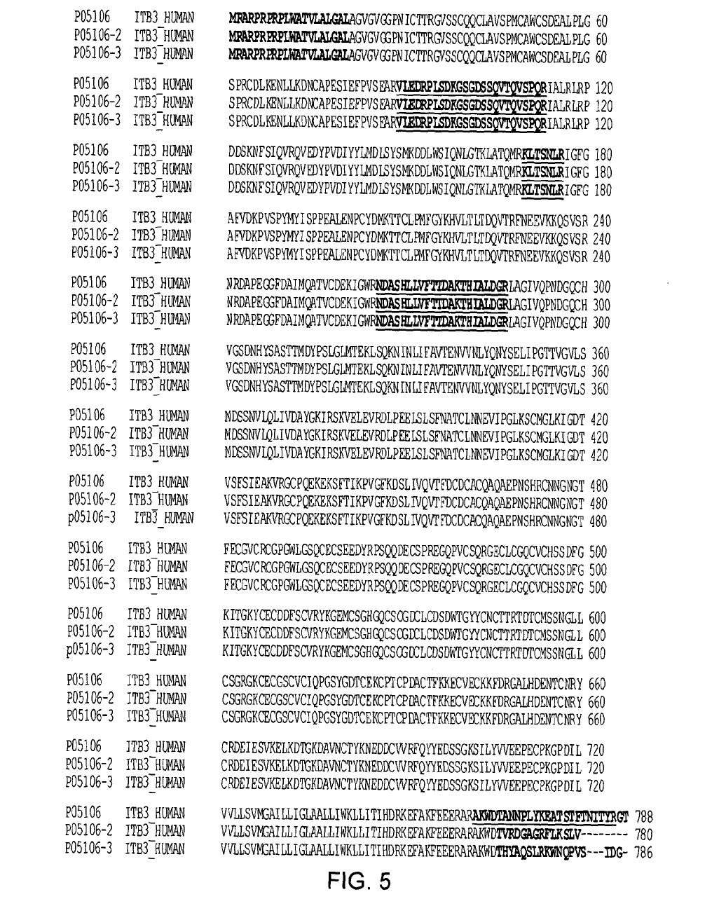

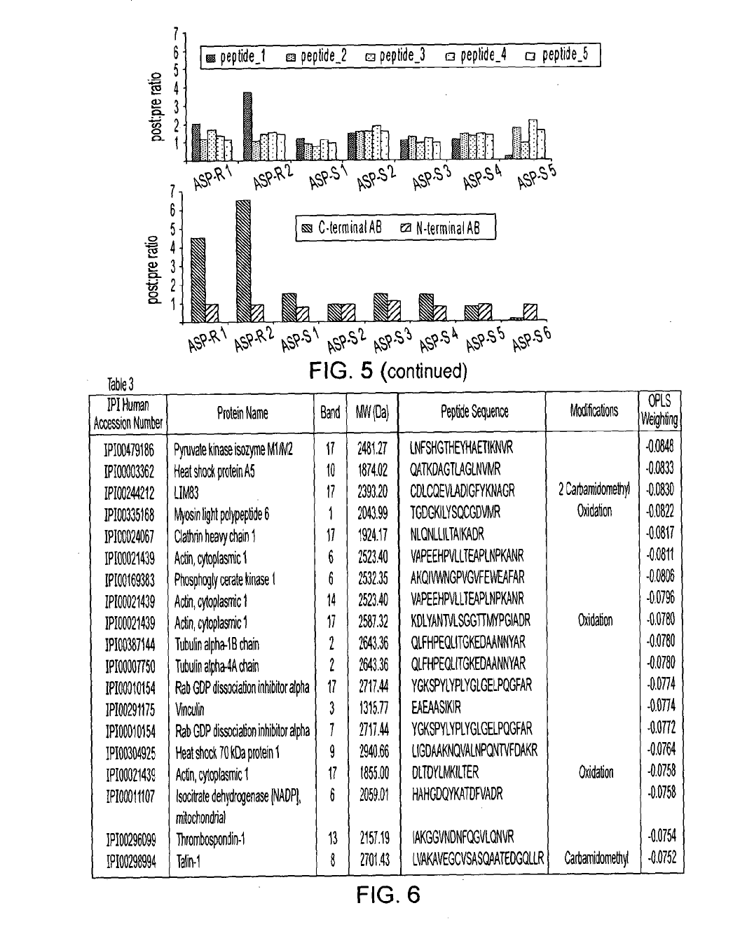

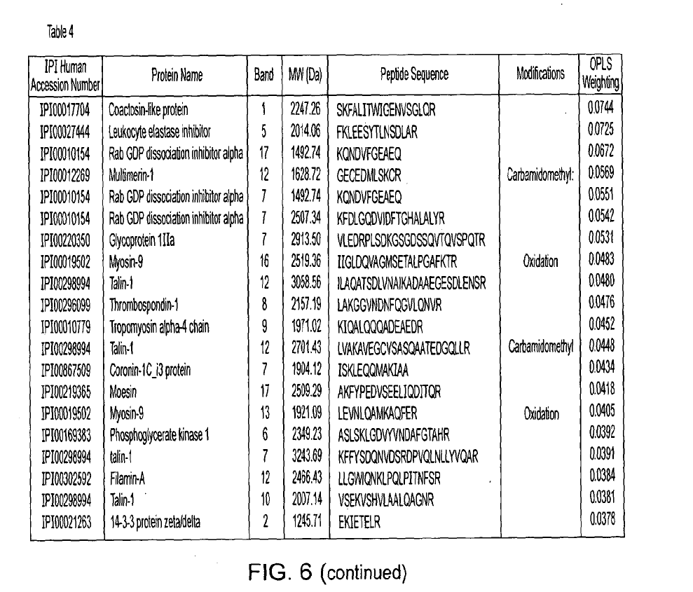

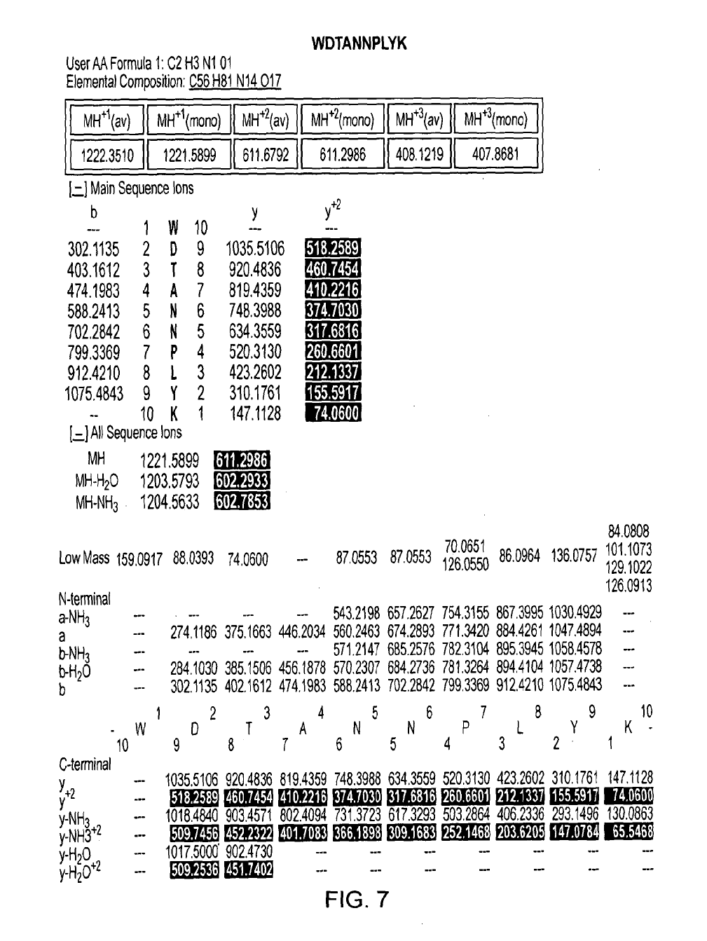

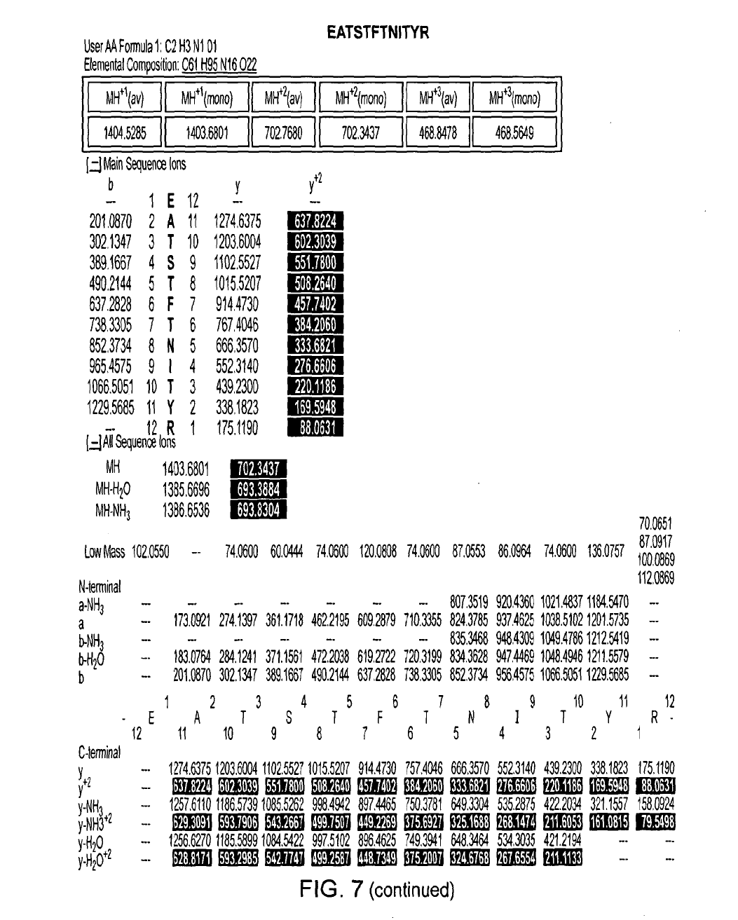

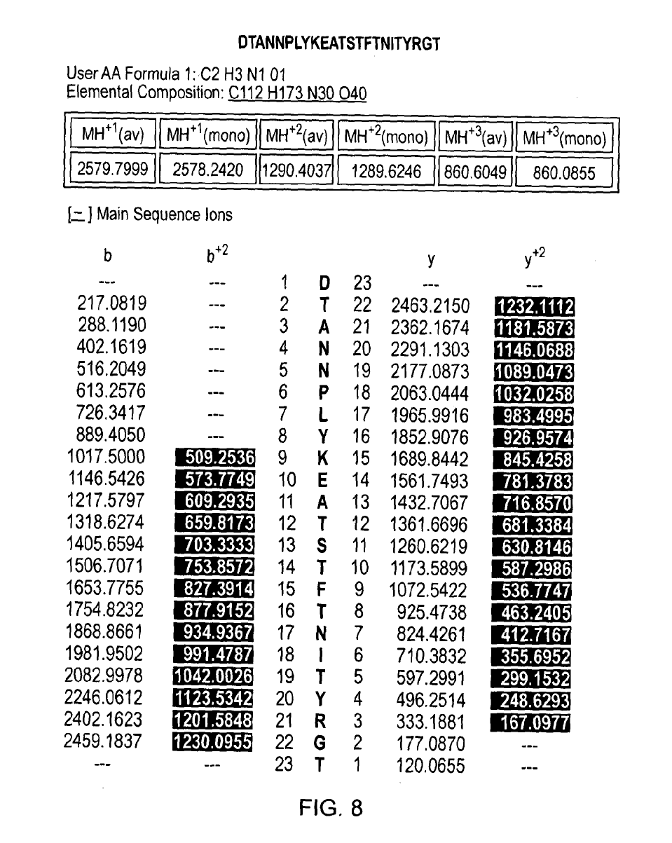

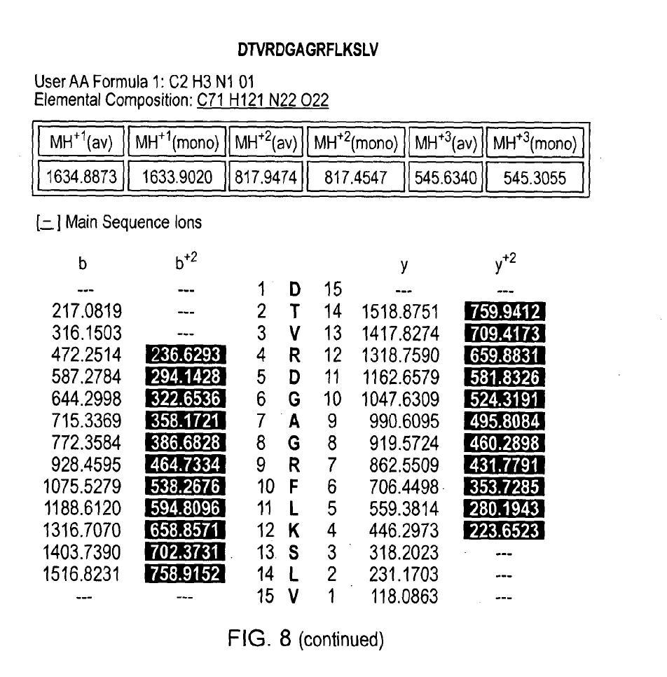

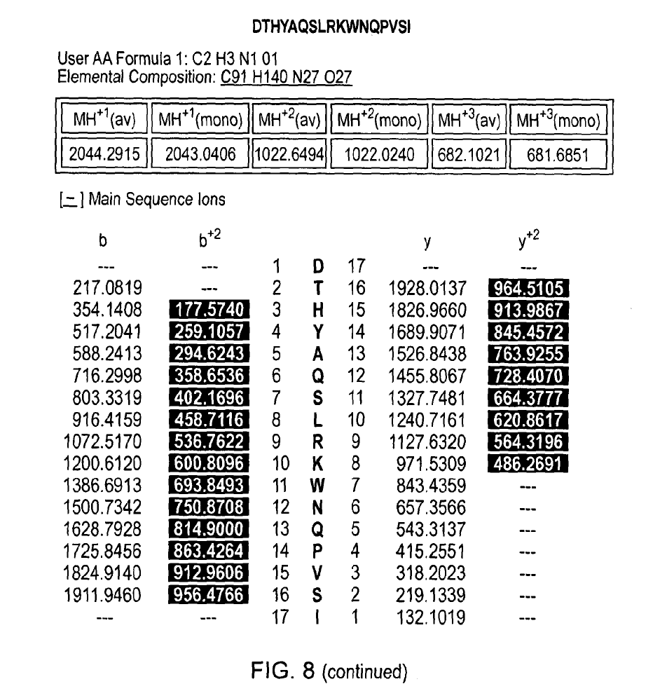

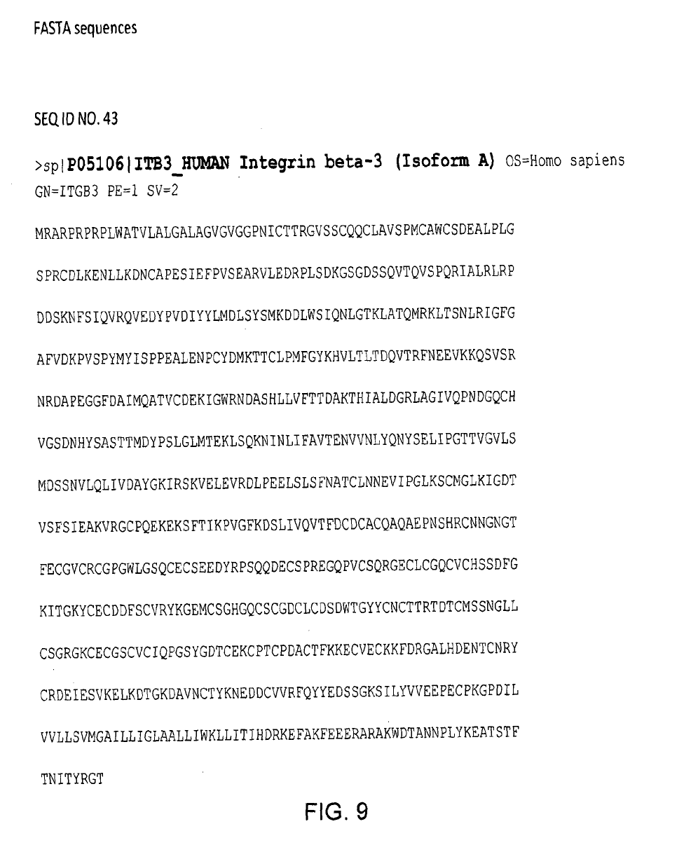

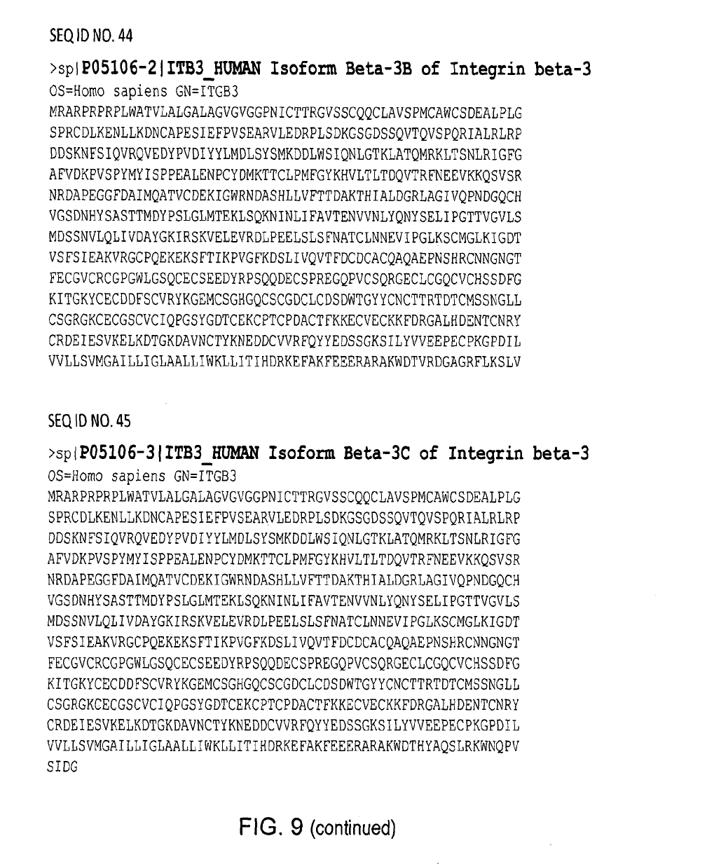

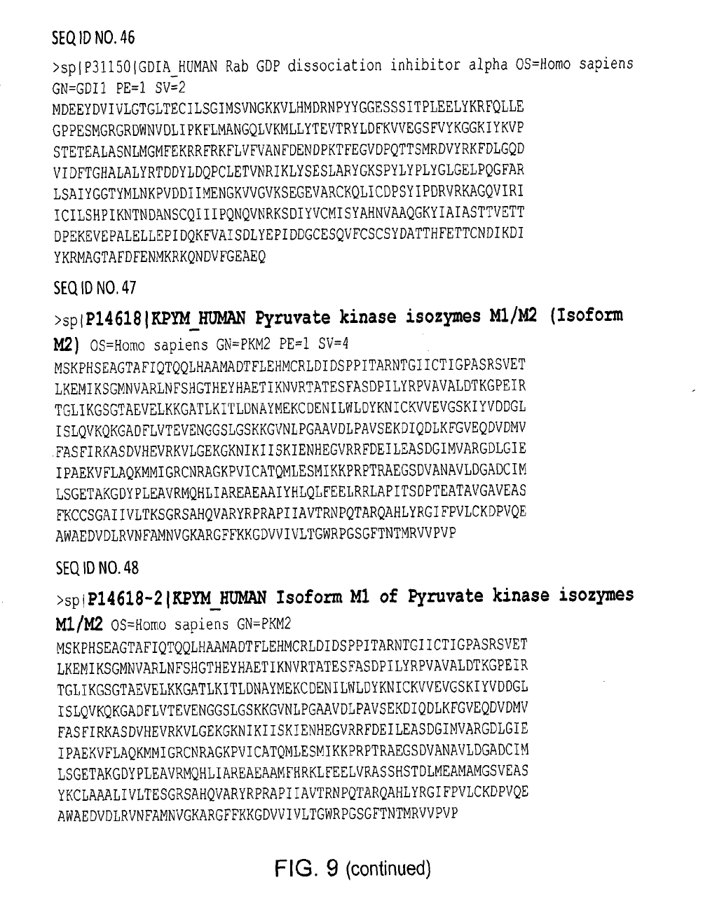

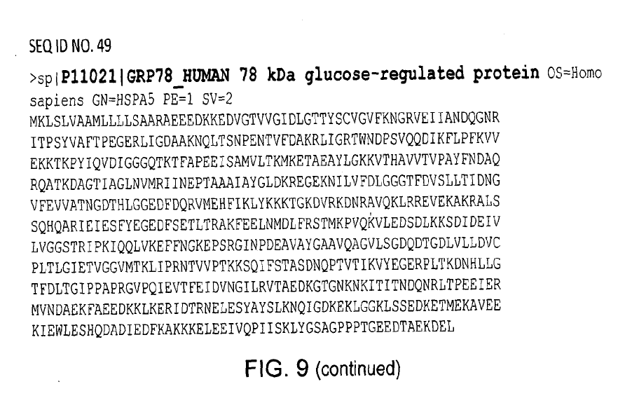

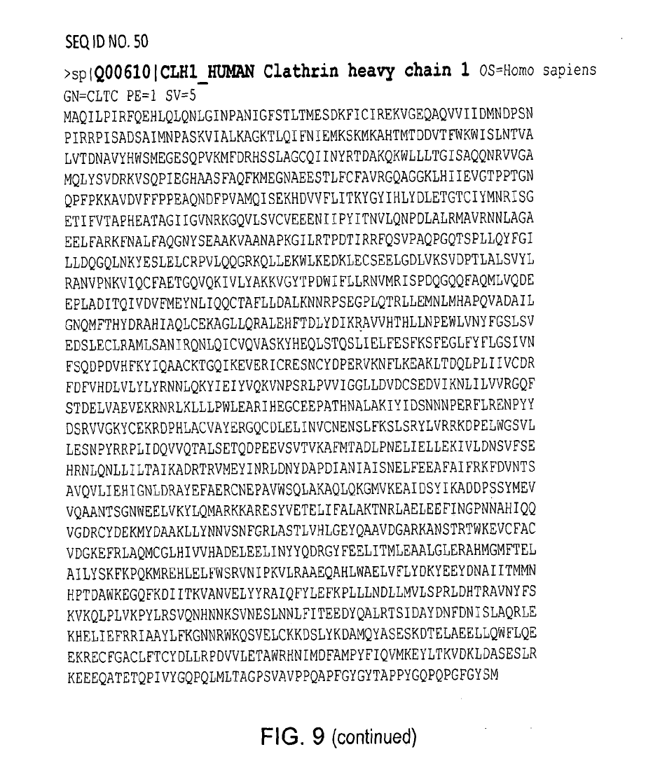

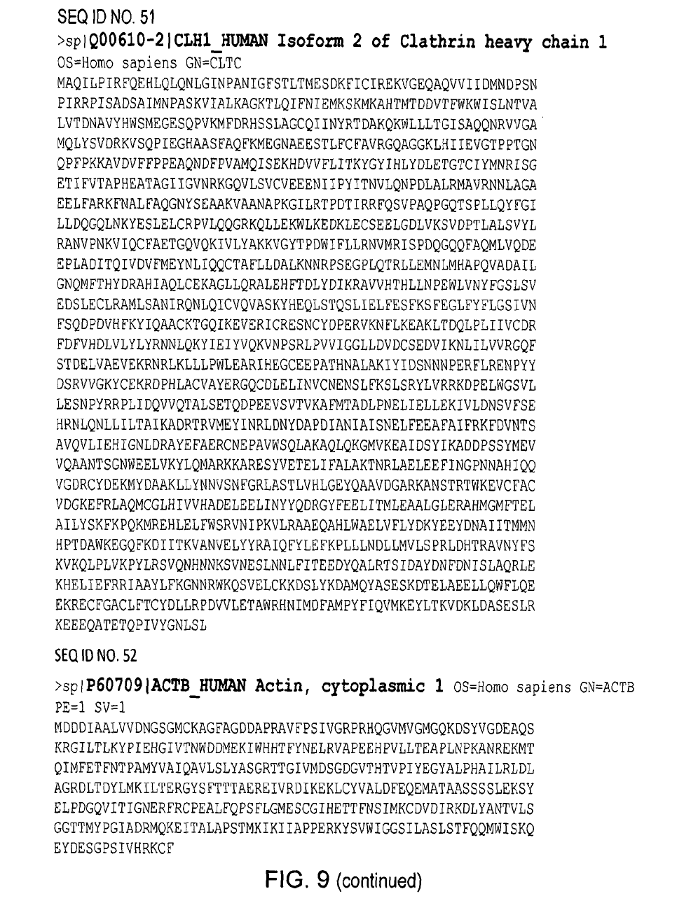

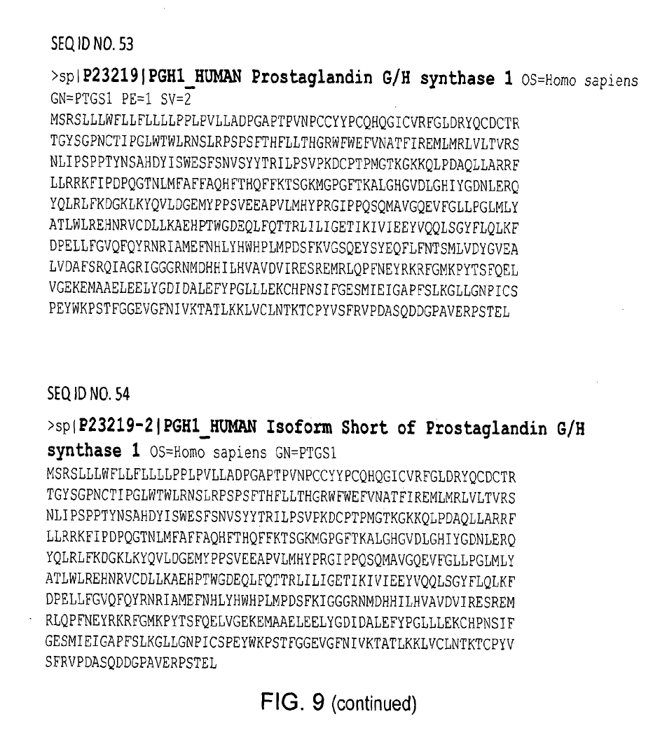

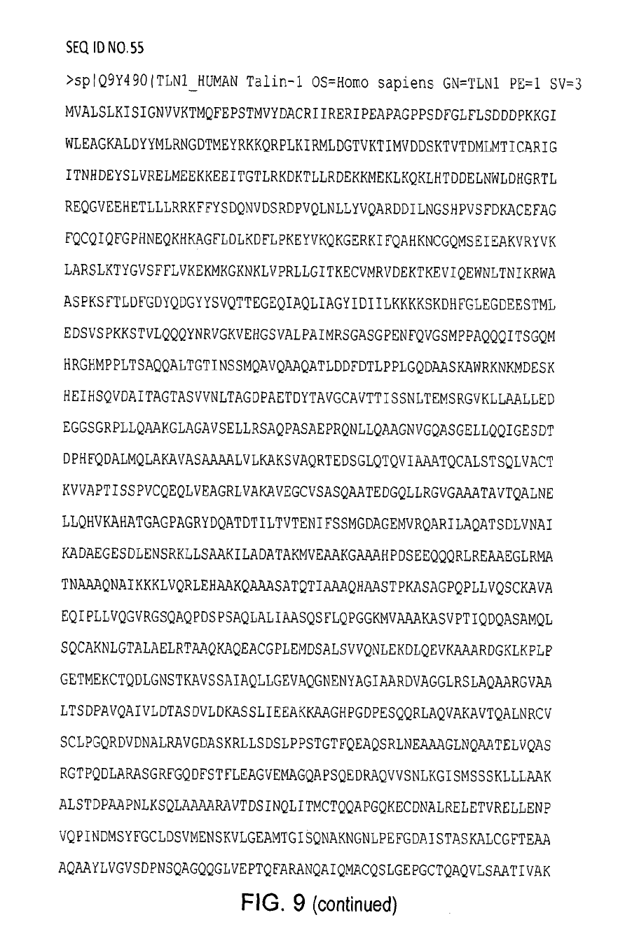

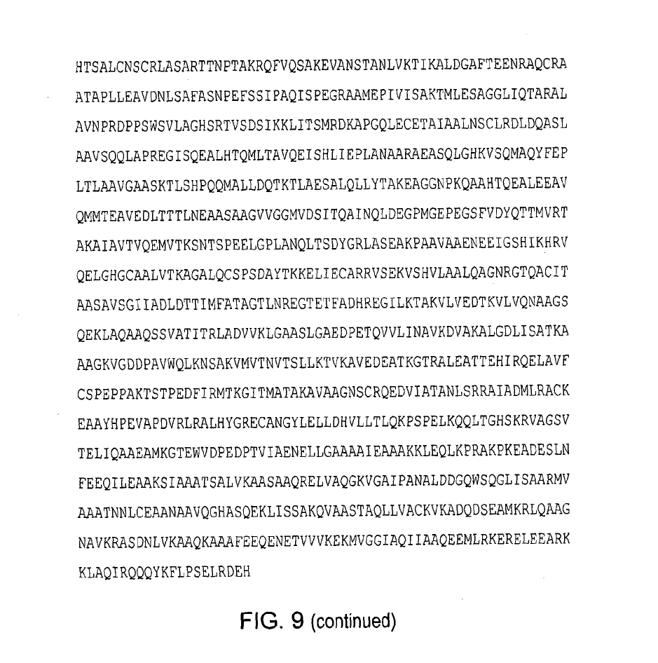

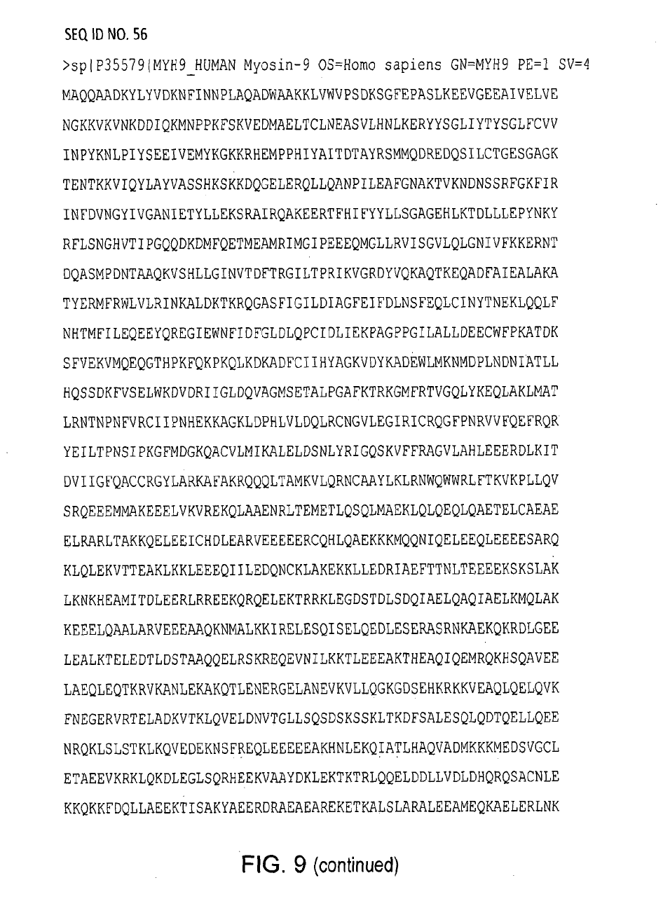

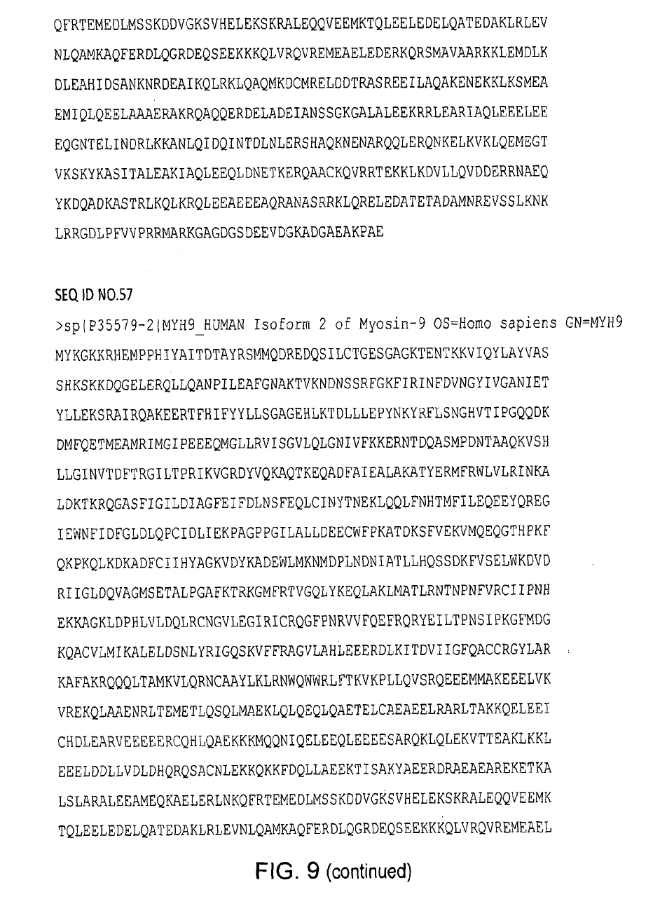

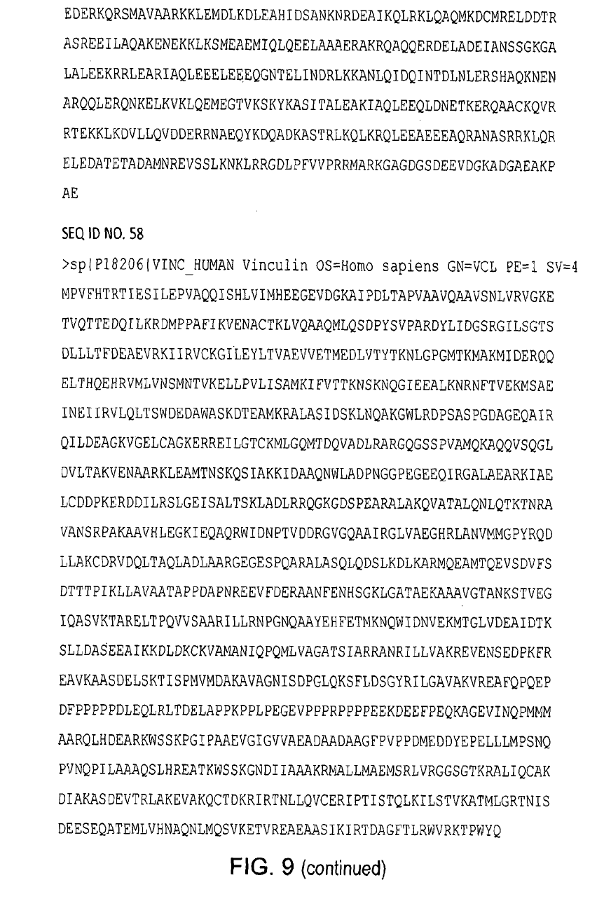

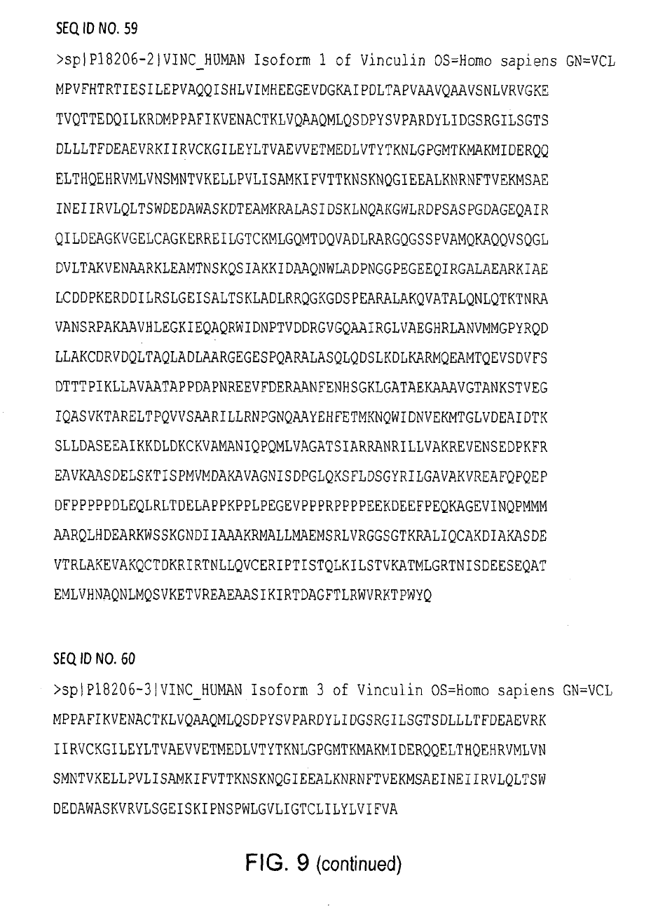

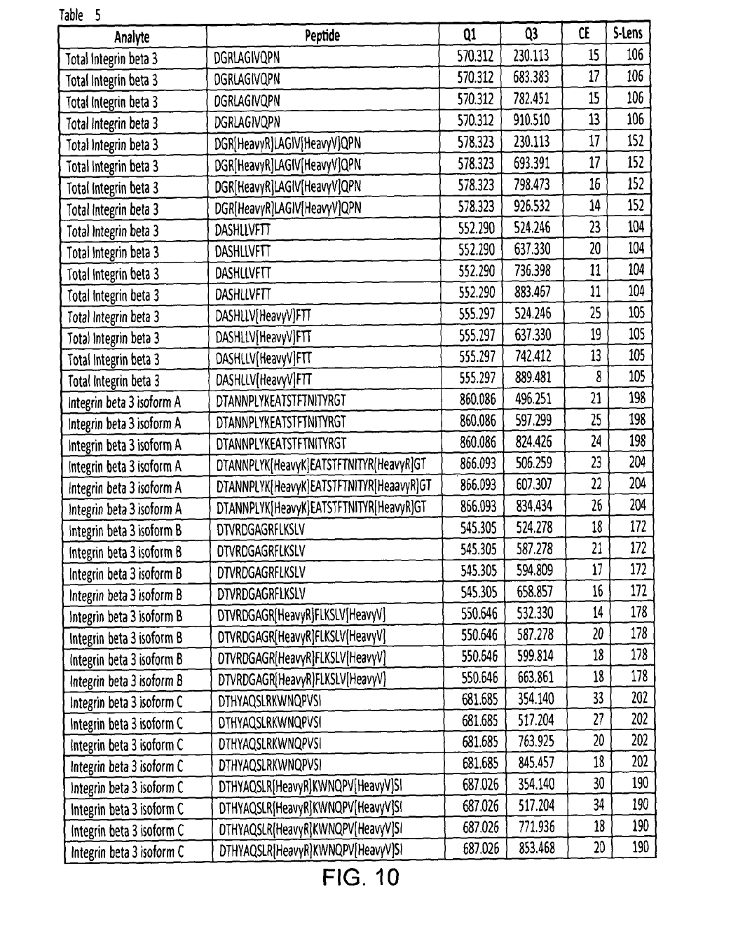

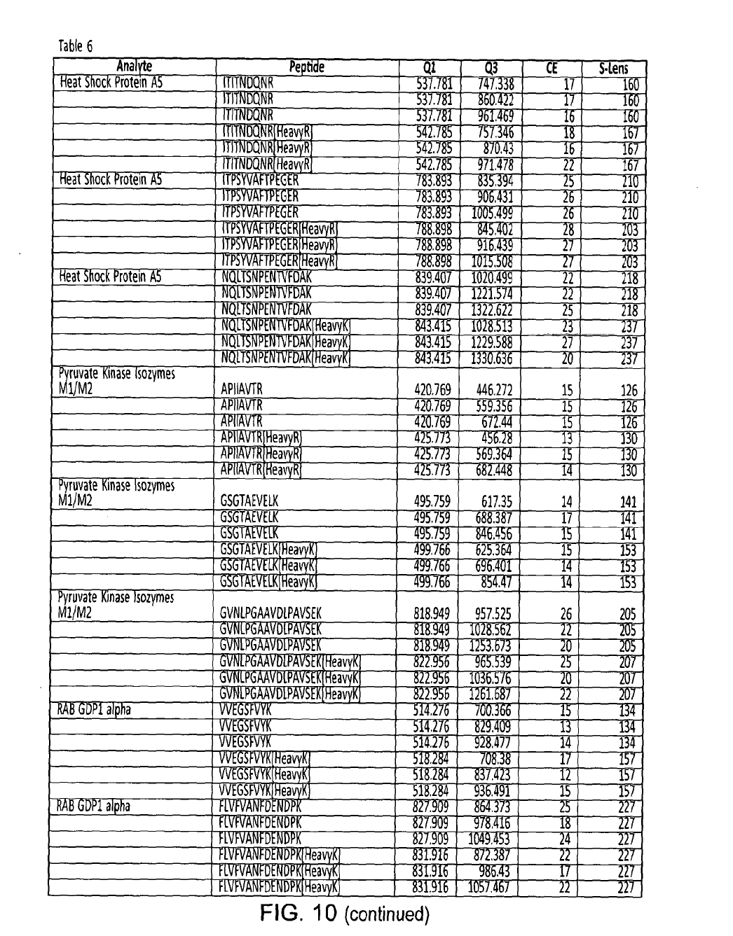

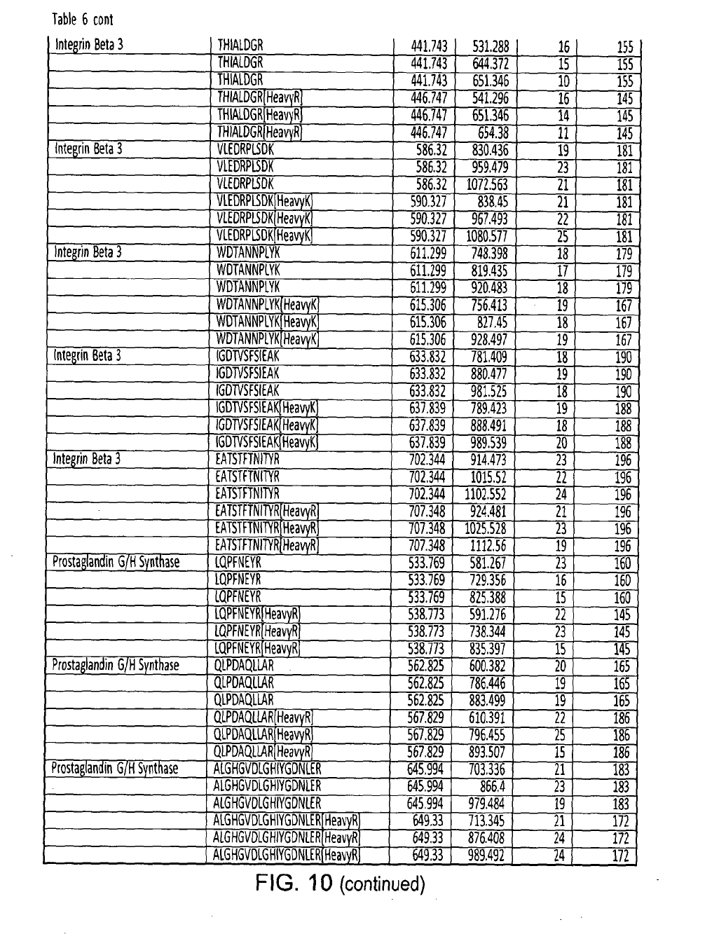

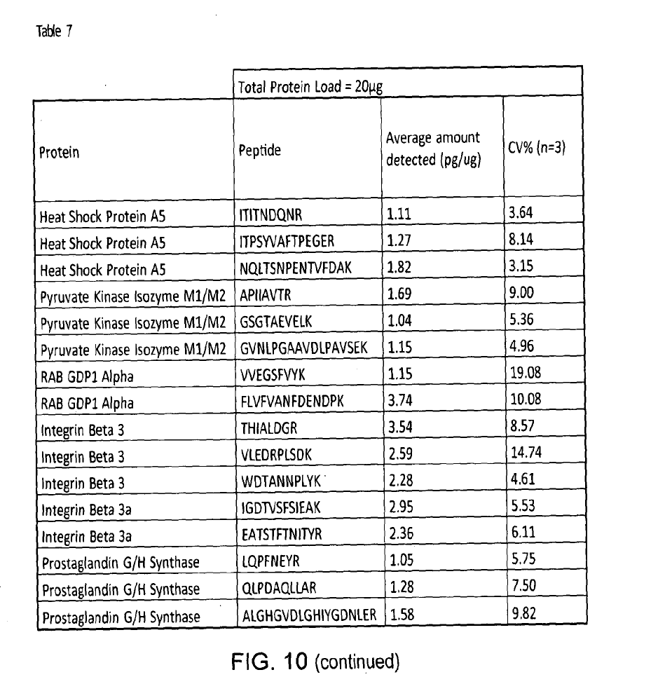

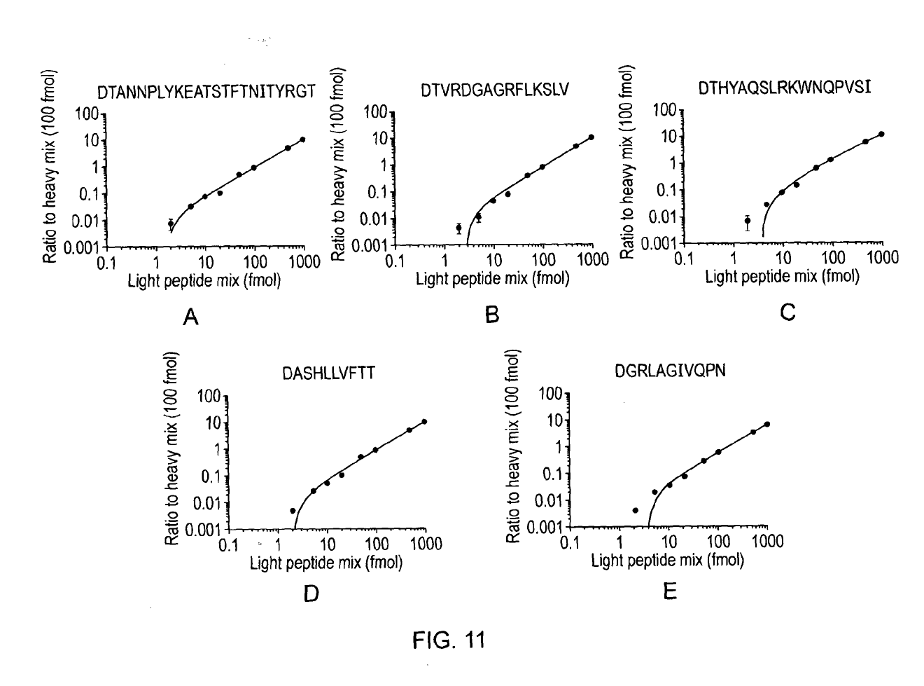

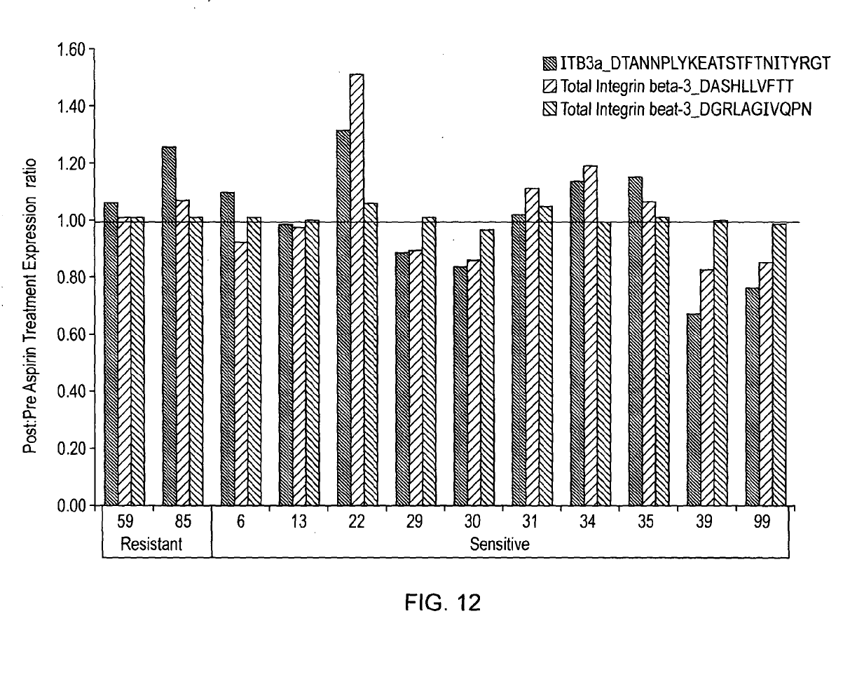

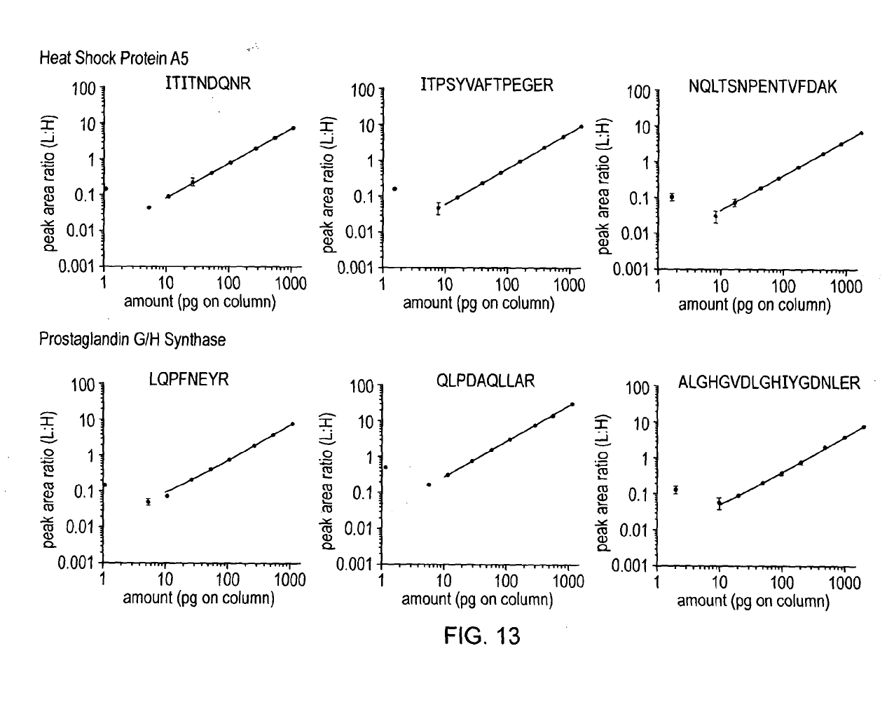

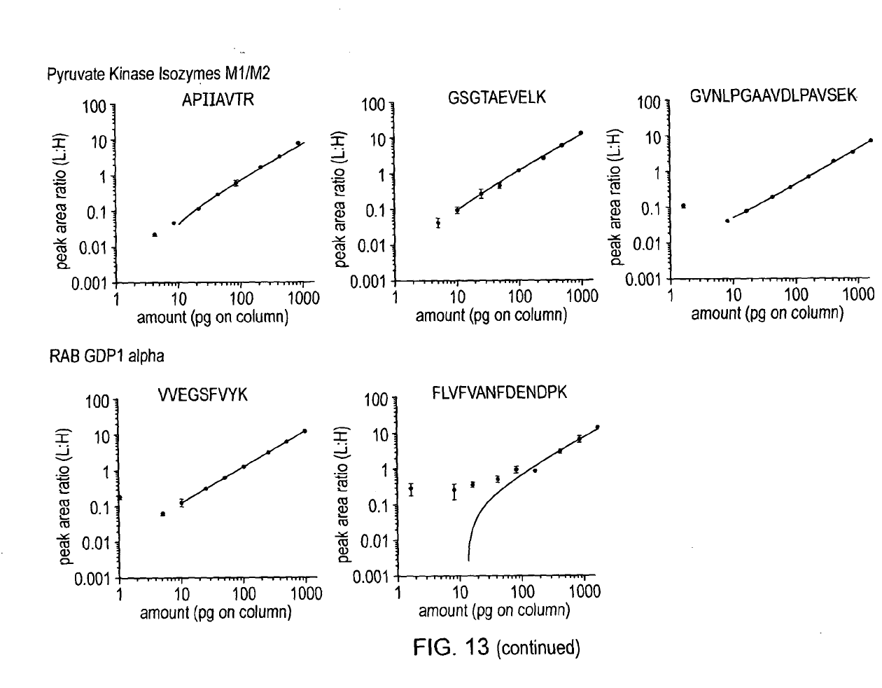

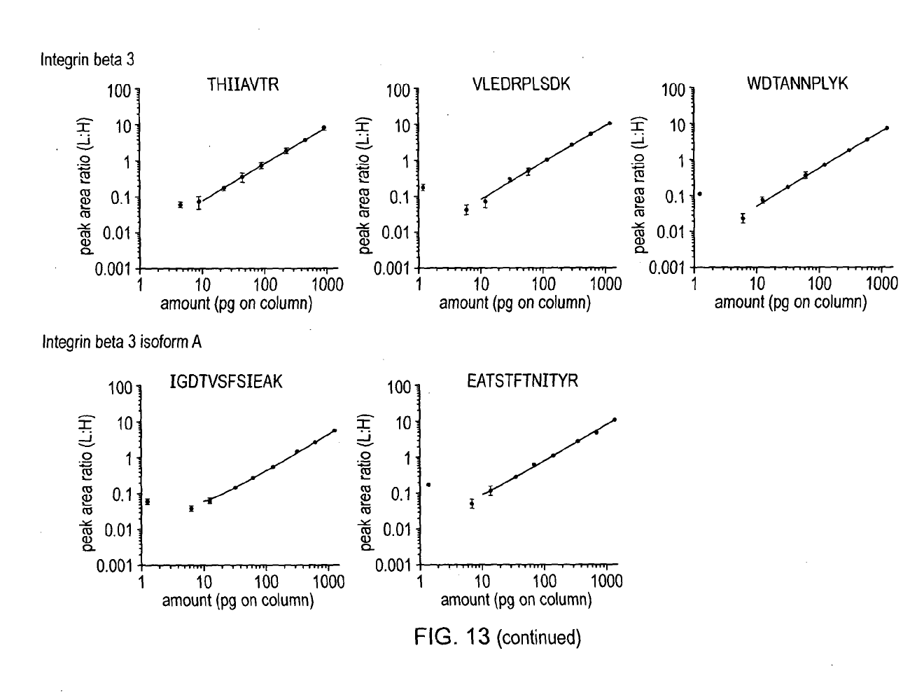

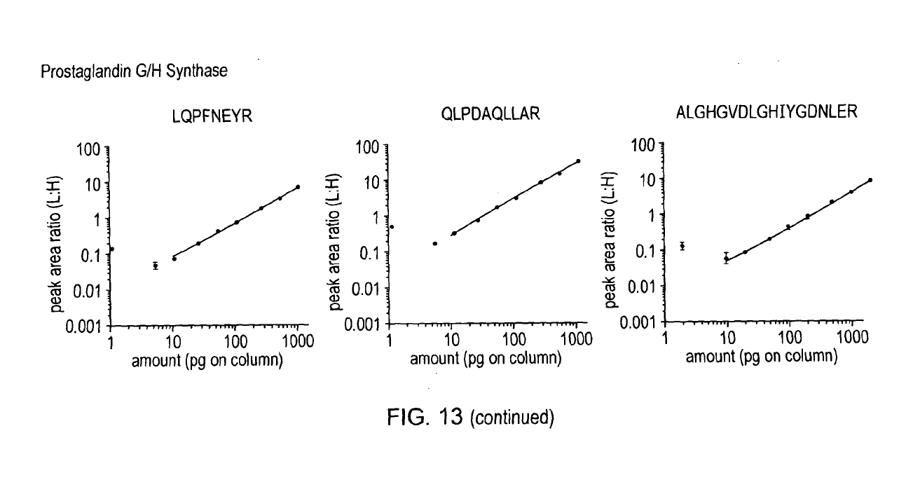

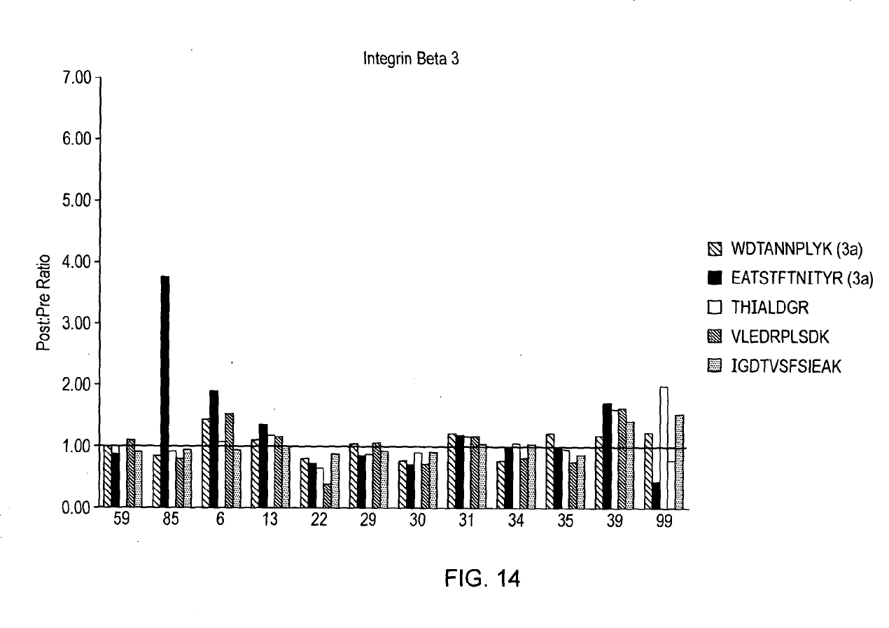

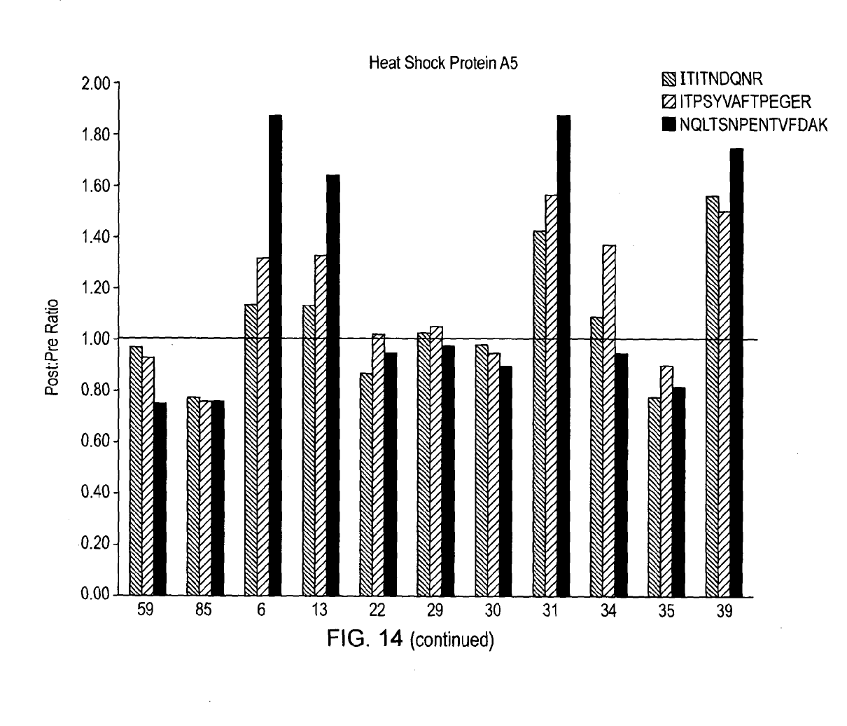

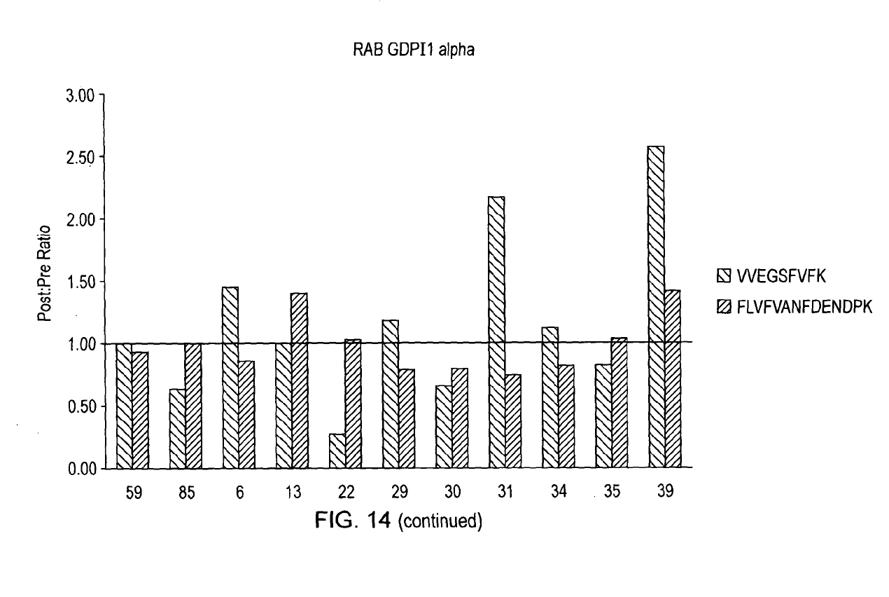

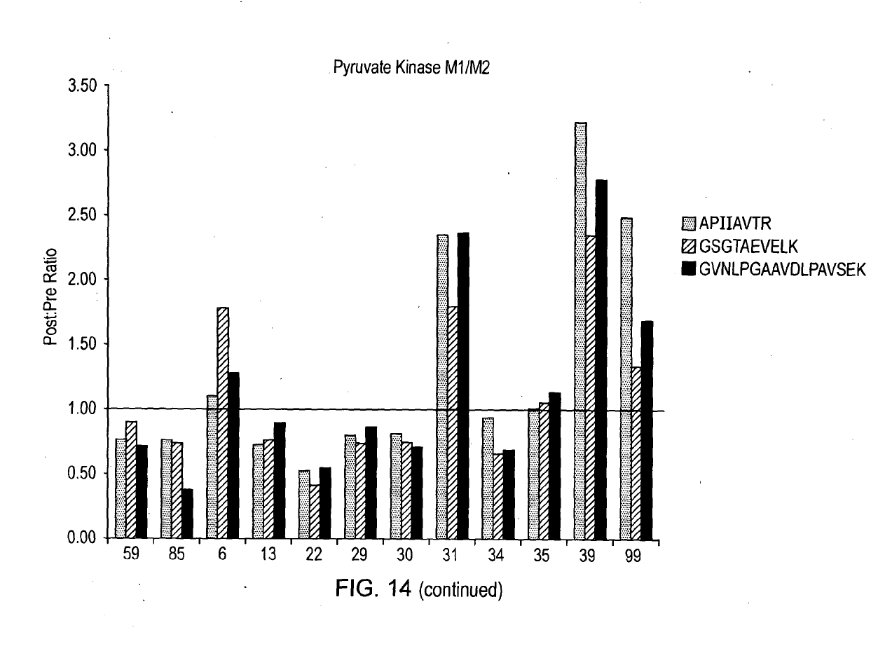

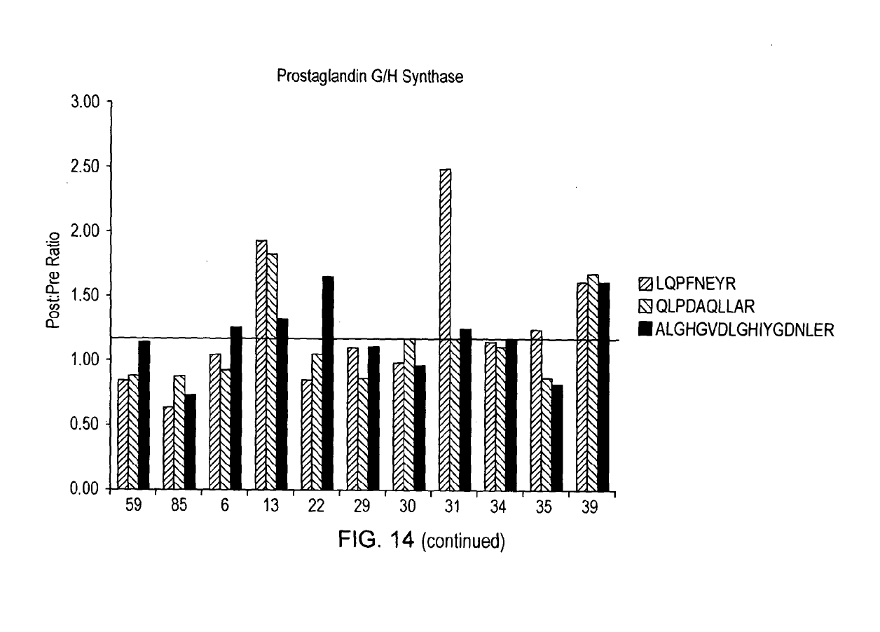

To reduce sample complexity and maximise the number of proteins identified in the discovery phase, labelled samples were mixed and analysed by GeLC-MS/MS. Briefly, separation of platelet lysates was performed by SDS-PAGE and each lane cut in 17 fractions. Each fraction was then subjected to in-gel digestion with Trypsin followed by reversed phase chromatographic separation feeding directly into a tandem mass spectrometer. This two-dimensional separation approach enables the identification and quantitation of a large number of proteins in the platelet proteome. (FIGS. 2 A and B).

From the discovery data four proteins were found to be differentially expressed in aspirin resistant compared to aspirin sensitive subjects after treatment with aspirin; clathrin-heavy chain-1, integrin beta 3, Rab GDI and pyruvate kinase.

These four proteins along with COX1 were taken forward for targeted analysis by SRM. COX1 peptides and fragment ions for SRM analysis were chosen based on in silico analysis using Pinpoint™ software (Thermo Scientific). Vinculin, myosin and talin are not influenced by aspirin treatment and were included as a constant reference to allow normalisation of the data between experiments.

In the absence of heavy-doped standard synthetic peptides, SRM method were designed based on the presence of time-aligned transitions. SRM transitions were selected either from discovery data and/or by in silico prediction using Pinpoint™ software. Initially, separate SRM methods for each protein were created (Myosin—21 transitions, 5 peptides; Talin-1—21 transitions, 5 peptides; Vinculin—6 peptides, 21 transitions; ITGB3—5 peptides, 20 transitions; Cox-1—7 peptides, 29 transitions; Pyruvate kinase—5 peptides, 18 transitions; Clathrin—3 peptides, 12 transitions; RAE GDP—4 peptides and 19 transitions, Table 2). Peptides that did not show consistant time-aligned transitions during the SRM development or which were not detectable in all subject samples were not included in the final method.

The final method contained 16 peptides with 64 transitions representing 8 proteins (Table 2 & FIG. 4). Targeted analysis of selected proteins by SRM was carried out in six subject samples pre and post treatment with aspirin which resulted in the quantitation of two candidate proteins; ITGB3 (4 peptides, 18 transitions) and RAB GDP (1 peptide, 4 transitions) and two normalisation proteins; vinculin (2 peptides, transitions) and talin-1 (3 peptides, 12 transitions). Following SRM analysis, surprisingly, only one ITGB3 peptide (AKWDTANNPLYKEATSTFTNITYR—SEQ ID NO. 1) specific for isoform beta 3A consistently showed significant changes in aspirin resistant compared to aspirin sensitive subjects. There are three isoforms of ITGB3 that vary in sequences at the C-terminal end of the protein (see FIG. 5a). SRM analysis of ITGB3 peptides which are common to all three isoforms showed no difference in protein levels after treatment with aspirin.

To further evaluate these findings, fresh aliquots of the same subject samples were analysed by Western Blotting, using two antibody preparations raised against the C-terminus and N-terminus of the protein respectively. The antibodies were raised against the N-terminal and the C-terminal 20 amino acids of ITGB3A. At the N-terminus this sequence is identical between all three isoforms of ITGB3 and therefore serves as a total measure of all isoforms, whereas the C-terminal region is highly unique to Isoform A allowing discrimination between levels of Isoform A and Isoforms B and C. This confirmed the SRM results showing that levels of ITGB3 isoform A are increased following aspirin treatment in resistant subjects compared to aspirin sensitive subjects (FIG. 5).

The identification of a C-terminal quantitative difference for ITGB3 isoform A is surprising. It has previously been shown in some studies that platelet resistance is associated with genetic polymorphisms of the platelet glycoprotein IIIc (ITGB3) gene. In particular Macchi et al. (2003. JACC, 42: 1115-1119) showed that the A1 polymorphism affecting the N-terminal region of ITGB3 was associated with platelet resistance after aspirin treatment. Conversely, Pamucku et al. (2005. Am. Heart J., 149: 675-680) found no such enrichment of the A1 allele in patients with coronary stents with aspirin resistance compared to a similar group whose platelets remained sensitive to aspirin treatment. Consequently, it is clear that the role of ITGB3 in platelet resistance remains unclear and that the discovery of a C-terminal peptide fragment suitable for the accurate determination of platelet resistance provides a novel approach for management of this clinically challenging phenomenon.

The results presented here show the determination and the evaluation of selected candidates for aspirin resistance by monitoring changes in protein expression pre to post aspirin treatment. Additionally, the presented experiments demonstrate the utility of TMT for both discovery based and evaluation based experiments, without the need for generation of new sample sets (FIG. 1).

Applied to the identification of differentially expressed proteins pre to post aspirin treatment comparing aspirin resistant and sensitive subjects several proteins were found to be regulated in patients with resistant platelets following aspirin treatment. For one of these proteins, ITGB 3 isoform A, a specific C-terminal fragment was seen to be up-regulated in aspirin resistant subjects.

1-56. (canceled)

57. A method of determining platelet sensitivity to an anti-platelet agent, said method comprising
determining the protein expression levels of integrin beta 3 in a platelet sample; said sample having been
(i) obtained from an individual previously treated with said anti-platelet agent; or
(ii) obtained from an individual and then contacted in vitro with said anti-platelet agent;
comparing said integrin beta 3 expression levels with one or more reference levels; and
determining platelet sensitivity to the anti-platelet agent for said sample based on differences in expression levels of integrin beta 3 as compared to the one or more reference levels.

58. A method according to claim 57 wherein integrin beta 3 is integrin beta 3 isoform A.

59. A method according to claim 57 wherein the step of determining the protein expression levels of integrin beta 3 includes determining the protein expression levels of integrin beta 3 isoform A, integrin beta 3 isoform B and/or integrin beta 3 isoform C respectively.

60. A method according to claim 59 wherein the step of determining the protein expression levels includes determining changes in expression levels integrin beta isoforms A, B and/or C relative to each other.

61. A method according to claim 60 wherein the expression levels of integrin beta 3 isoforms A, B and/or C are determined using a binding member specific for each isoform.

62. A method according to claim 61 wherein said binding member for integrin beta 3 isoform A is capable of specifically binding to the amino acid sequence AKWDTANNPLYKEATSTFTNITYR (SEQ ID NO.1), or to a nucleic acid molecule comprising a nucleic acid sequence encoding amino acid sequence AKWDTANNPLYKEATSTFTNITYR (SEQ ID NO.1).

63. A method according to claim 61 wherein said binding member is a nucleic acid sequence capable of hybridising with a nucleic acid molecule comprising sequence encoding amino acid sequence selected from the group consisting of
integrin beta 3 isoform A (IGB3A)
(SEQ ID NO. 1)
AKWDTANNPLYKEATSTFTNITYR,
(SEQ ID NO. 2)
AKWDTANNPLYKEATSTFTNITYRGT;
integrin beta 3 isoform B (IGB3B)
(SEQ ID NO. 3)
AKWDTVRDGAGRFLKSLV;
and
integrin beta 3 isoform C (IGB3C)
(SEQ ID NO. 4)
AKWDTHYAQSLRKWNQPV.

64. A method according to claim 61 wherein said isoform specific binding member is an antibody.

65. A method according to claim 57 wherein the anti-platelet agent is aspirin.

66. A method according to claim 57 wherein the step of determining the protein expression levels of integrin beta 3 in the platelet sample comprises
digesting said proteins to produce a population of peptides;
determining the abundance of one or more of said peptides derived from integrin beta 3 using Selected Reaction Monitoring; and
wherein the comparing step includes comparing the abundance of said one or more peptides with a pre-determined peptide abundance associated with anti-platelet resistance; and
wherein determining platelet sensitivity is based on the differences in abundance of said one or more peptides.

67. A method according to claim 66 wherein the pre-determined peptide abundance is determined using a known amount of corresponding synthetic peptides selected from Table 2.

68. A method according to claim 66 wherein the pre-determined peptide abundance is determined using a peptide population obtained from said individual post anti-platelet treatment.

69. A method according to claim 68 wherein said one or more peptides are selected from SEQ ID NOs. 18 to 20.

70. A method according to claim 66 further comprising determining the abundance of one or more peptides derived from one or more marker proteins selected from Table 3, Table 4, Table 5 and Table 6.

71. A method according to claim 66 wherein said one or more peptides are determined using a known amount of corresponding synthetic peptides selected from the group consisting of
Myosin
SEQ ID NO. 5
LK*K*ANLQIDQINTDLNLER
SEQ ID NO. 6
EK*QLAAENR
SEQ ID NO. 7
DELADEIANSSGK*GALALEEK*R
SEQ ID NO. 8
INFDVNGYIVGANIETYLLEK*SR
Talin-1
SEQ ID NO. 9
ALEATTEHIR
SEQ ID NO. 10
DPPSWSVLAGHSR
SEQ ID NO. 11
VSEK*VSHVLAALQAGNR
SEQ ID NO. 12
LAQVAK*AVTQALNR
SEQ ID NO. 13
K*FFYSDQNVDSR
Vinculin
SEQ ID NO. 14
EAEAASIK*IR
SEQ ID NO. 15
GILSGTSDLLLTFDEAEVR
SEQ ID NO. 16
SLGEISALTSK*LADLR
SEQ ID NO. 17
DPSASPGDAGEQAIR
ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN
COX-1
SEQ ID NO. 21
VCDLLK*AEHPTWGDEQLFQTTR
SEQ ID NO. 22
VPDASQDDGPAVER
SEQ ID NO. 23
VPDASQDDGPAVERSTEL
SEQ ID NO. 24
WFWEFVNATFIR
SEQ ID NO. 25
LQPFNEYR
Pyruvate kinase
SEQ ID NO. 26
LDIDSPPITAR
SEQ ID NO. 27
LNFSHGTHEYHAETIK*NVR
SEQ ID NO. 28
GIFPVLCK*DPVQEAWAEDVDLR
SEQ ID NO. 29
TATESFASDPILYRPVAVALDTK*GPEIR
SEQ ID NO. 30
EAEAAIYHIQLFEELR
Clathrin
SEQ ID NO. 31
LK*LLLPWLEAR
SEQ ID NO. 32
K*DPELWGSVLLESNPYR
SEQ ID NO. 33
NLQNLLILTAIK*ADR
RAB GDP
SEQ ID NO. 34
K*FDLGQDVIDFTGHALALYR
SEQ ID NO. 35
YGK*SPYLYPLYGLGELPQGFAR
SEQ ID NO. 36
NPYYGGESSSITPLEELYK*R,
and
SEQ ID NO. 37
K*QNDVFGEAEQ;
wherein symbol K* represents a modified lysine residue bearing an isotopic tag or isobaric tag.

72. A method according to claim 71 wherein said synthetic peptides are selected from the group consisting of
Myosin
SEQ ID NO. 5
LK*K*ANLQIDQINTDLNLER
Talin-1
SEQ ID NO. 9
ALEATTEHIR
SEQ ID NO. 10
DPPSWSVLAGHSR
SEQ ID NO. 11
VSEK*VSHVLAALQAGNR
Vinculin
SEQ ID NO. 14
EAEAASIK*IR
SEQ ID NO. 15
GILSGTSDLLLTFDEAEVR
ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN
RAB GDP
SEQ ID NO. 34
K*FDLGQDVIDFTGHALALYR;
wherein symbol K* represents a modified lysine residue bearing an isotopic tag or isobaric tag.

73. A method according to claim 72 wherein the synthetic peptides are selected from the group consisting of
ITGB3
SEQ ID NO. 18
K*LTSNLR
SEQ ID NO. 19
VLEDRPLSDK*THIALDGR
ITGB3A
SEQ ID NO. 38
WDTANNPLYK
SEQ ID NO. 39
EATSTFTNITYR
SEQ ID NO. 40
DTANNPLYKEATSTFTNITYRGT
SEQ ID NO. 20
AK*WDTANNPLYK*EATSTFTNITYR
ITGB3B
SEQ ID NO. 41
DTVRDGAGRFLKSLV
ITGB3C
SEQ ID NO. 42
DTHYAQSLRKWNQPVSI
Shared ITGB3
SEQ ID NO. 43
DASHLLVFTT
SEQ ID NO. 44
DGRLAGIVQPN;
wherein symbol K* represents a modified lysine residue bearing an isotopic tag or isobaric tag.

74. A method of monitoring cardiovascular disease or cerebrovascular disease in an individual undergoing treatment with an anti-platelet agent, said method comprising determining the expression profile in a platelet sample of integrin beta 3 isoforms A B and/or C.

75. A method according to claim 74 wherein said method uses SEQ ID NOs 18, 19 or 20 in a Selected Reaction Monitoring assay using one or more of the transitions listed in Table 2.

76. A method according to claim 74 wherein the cardiovascular disease is ischaemic heart disease.

77. A method according to 74 wherein the cerebrovascular diseases is transient ischaemic attack or ischaemic stroke.
